(19) United States
(12) Patent Application Publication
(10) Pub. No.: US 2009/0227464 A1
(43) Pub. Date: Sep. 10, 2009

(54) Prognosis determination in ewing sarcoma patients by means of genetic profiling

(76) Inventors: Smadar Avigad, Ramat Gan (IL); Isaac Yaniv, Petach Tikva (IL); Rina Zaizov, Herzelia Pituach (IL); Haim Marx, Herzelia Pituach (IL); Anat Ohali, Ramat Gan (IL)

Correspondence Address: HESLIN ROTHENBERG FARLEY & MESITI PC, 5 COLUMBIA CIRCLE, ALBANY, NY, 12203, US

(21) Appl. No.: 10/562,527

(22) PCT Filed: Jun. 30, 2004
(86) PCT No.: PCT/IL04/00578

§ 371 Date: Apr. 23, 2007

Related U.S. Application Data
(60) Provisional application No. 60/483,626, filed on Jul. 1, 2003.

(51) Int. Cl. C40B 30/04; C12Q 1/68; G01N 33/53; C40B 40/08

(52) U.S. Cl. 506/9; 435/6; 435/7.92; 506/17

(57) ABSTRACT

The present invention provides a method for assessing the prognosis of Ewing's Sarcoma patients comprising determining the expression pattern of a defined set of genes in tumor material obtained from said patients, and assigning said expression pattern to either a good prognosis or poor prognosis group.

### FIELD OF THE INVENTION

The present invention relates to a method for assessing prognosis in cancer patients. More specifically, the invention disclosed hereinbelow provides a genetic analysis technique that may be used to assess the prognosis of patients with Ewing Sarcoma.

### BACKGROUND OF THE INVENTION

Ewing's Sarcoma (ES) is the second most common primary malignant bone tumor in children and adolescents and it belongs to a group of neuroectodermal tumors known as Ewing's Sarcoma Family of Tumors (EFT). This is an aggressive tumor with a high propensity for recurrence and distant metastases [Ginsberg, J. P. et al. “Ewing sarcoma family of tumors: Ewing's sarcoma of bone and soft tissue and the peripheral primitive neuroectodermal tumors.” In: Principles and Practice of Pediatric Oncology, (eds.: Pizzo, P. A. & Poplack) 4th edition, 973-1016, Philadelphia, Pa., 2002].

All EFT share specific translocations resulting in the fusion of the EWS gene on chromosome 22q12 with different ETS oncogenes on different chromosomes; the most frequent (˜95%) is FLI1 on chromosome 11. These translocations are considered distinct diagnostic features of ES tumors [Delattre, O. et al., New Eng. J. Med. 331, 294-299 (1994)].

Both the primary site of the tumor, and the initial response to therapy (assessed histologically as the degree of tumor necrosis following surgery), have become accepted valid prognostic factors in localized tumors. In spite of advances in multimodal therapy, including combination of aggressive chemotherapy, radiotherapy and surgery, about 50% of patients eventually relapse, even after 5 years [Terrier, P. et al., Semin. Diagn. Pathol. 13, 250-257

Current clinical and biological characteristics fail to accurately classify ES patients according to their clinical behavior, and it is therefore essential to search for novel reliable prognostic parameters, already at diagnosis.

It is therefore a purpose of the present invention to provide a genetic profiling method for prognosis assessment of patients presenting with ES.

It is another purpose of the invention to provide materials and kits for performing the aforementioned method.

Further objects and advantages of the present invention will become apparent as the description proceeds.

### SUMMARY OF THE INVENTION

It has now been found that it is possible to distinguish between ES patients having a good prognosis and those having a poor prognosis by means of comparing gene expression patterns in nucleic acid material isolated from the tumors of said patients. Furthermore, it has been found that this prognosis determination may be performed very early on, during initial diagnosis.

The present invention is primarily directed to a method for assessing the prognosis of ES patients comprising determining the expression pattern of a defined set of genes in tumor material obtained from said patients, and assigning said expression pattern to either a good prognosis or poor prognosis group.

The term “good prognosis” is used herein to indicate that the patients are not expected to show ES-related signs, symptoms or evidence for a period of time compatible with the usual clinical meaning of the term. In many cases, this may be taken to mean that the patient is expected to be free from ES-related symptoms for at least five years from assessment. The term “poor prognosis” is similarly used to indicate that the patients are expected to relapse during treatment or within the first few years following treatment.

The term “expression pattern” is used herein to refer to the overall profile of results obtained when the expression of a defined set of genes is determined. Such a pattern is advantageous since it facilitates the use of both quantitative, statistical analytical techniques as well as permitting rapid visual inspection and comparison of results. Preferably (but not exclusively) such a pattern is obtained by the use of a matrix method, such as a high density microarray method.

Although any suitable technique may be used to determine the expression of the aforementioned defined set of genes, in one preferred embodiment of the method, this technique is a nucleic acid hybridization technique.

In a particularly preferred embodiment, the nucleic acid hybridization technique comprises the steps of extracting total RNA from the ES-patient tumor material, generating double-stranded cDNA from said total RNA, performing in vitro transcription of said cDNA, labeling the RNA transcript obtained thereby, preparing a hybridization mix comprising said labeled RNA transcript together with irrelevant and control nucleic acid sequences, hybridization of said hybridization mix to a solid-state human genome microarray and generating and amplifying a hybridization signal. This hybridization signal provides a visual expression pattern which may then be assigned to one of the good or poor prognosis groups.

In another preferred embodiment, the hybridization technique used is selected from the group consisting of northern blotting and western blotting.

In other preferred embodiments of the invention, gene expression may be determined by the use of a technique other than a hybridization technique. In a particularly preferred embodiment, the technique is selected from the group consisting of RT-PCR, semi-quantitative RT-PCR, quantitative real time RT-PCR, immunohistochemistry and ELISA.

In one particularly preferred embodiment of the method of the invention, the assignment of the gene expression pattern to one of the good or poor prognosis groups is performed by means of a hierarchical clustering technique.

In one preferred embodiment of the method of the invention, the aforementioned defined set of genes comprises genes selected from the group of 818 genes listed in table 1, hereinbelow.

In another preferred embodiment, the defined set of genes consists of between 1 and 100 genes selected from the aforementioned group of 818 genes.

In another preferred embodiment, the defined set of genes consists of between 101 and 200 genes selected from the aforementioned group of 818 genes.

In another preferred embodiment, the defined set of genes consists of between 201 and 300 genes selected from the aforementioned group of 818 genes.

In another preferred embodiment, the defined set of genes consists of between 301 and 400 genes selected from the aforementioned group of 818 genes.

In another preferred embodiment, the defined set of genes consists of between 401 and 500 genes selected from the aforementioned group of 818 genes.

In another preferred embodiment, the defined set of genes consists of between 501 and 600 genes selected from the aforementioned group of 818 genes.

In another preferred embodiment, the defined set of genes consists of between 601 and 700 genes selected from the aforementioned group of 818 genes.

In another preferred embodiment, the defined set of genes consists of between 701 and 818 genes selected from the aforementioned group of 818 genes.

In another aspect, the present invention is also directed to a solid-state nucleic acid microarray comprising at least two nucleic acids affixed to a substrate, wherein each of said at least two nucleic acids consists of a partial sequence of one of the genes present in the aforementioned group of 818 genes.

In one preferred embodiment, the microarray of the present invention comprises between 2 and 100 nucleic acid sequences, wherein each of said sequences consists of a partial sequence of one of the genes present in the aforementioned group of 818 genes.

In another preferred embodiment, the microarray of the present invention comprises between 101 and 200 nucleic acid sequences, wherein each of said sequences consists of a partial sequence of one of the genes present in the aforementioned group of 818 genes.

In another preferred embodiment, the microarray of the present invention comprises between 201 and 300 nucleic acid sequences, wherein each of said sequences consists of a partial sequence of one of the genes present in the aforementioned group of 818 genes.

In another preferred embodiment, the microarray of the present invention comprises between 301 and 400 nucleic acid sequences, wherein each of said sequences consists of a partial sequence of one of the genes present in the aforementioned group of 818 genes.

In another preferred embodiment, the microarray of the present invention comprises between 401 and 500 nucleic acid sequences, wherein each of said sequences consists of a partial sequence of one of the genes present in the aforementioned group of 818 genes.

In another preferred embodiment, the microarray of the present invention comprises between 501 and 600 nucleic acid sequences, wherein each of said sequences consists of a partial sequence of one of the genes present in the aforementioned group of 818 genes.

In another preferred embodiment, the microarray of the present invention comprises between 601 and 700 nucleic acid sequences, wherein each of said sequences consists of a partial sequence of one of the genes present in the aforementioned group of 818 genes.

In another preferred embodiment, the microarray of the present invention comprises between 701 and 818 nucleic acid sequences, wherein each of said sequences consists of a partial sequence of one of the genes present in the aforementioned group of 818 genes.

In a particularly preferred embodiment, the microarray of the present invention comprises all of the 818 genes present in the aforementioned group of genes.

In addition to the aforementioned at least two nucleic acids, the microarray may also comprise one or more control nucleic acid sequences.

The substrate present in the microarray may consist of any suitable material or combination of materials. Preferably, however, the substrate is selected from the group consisting of ceramics, glasses, metal oxides, nitrocellulose and nylon.

In a further aspect, the present invention also provides a kit comprising a solid-state nucleic acid microarray as defined and described herein together with an instruction sheet.

Kits based on the other gene expression technologies used in the method of the invention (as described hereinabove) are also within the scope of the present invention. Thus, in one embodiment, the kit of the present invention comprises a set of relevant primers suitable for use in real time RT-PCR together with control solutions and an instruction sheet. In another embodiment, the kit comprises micro-well plates or similar vessels suitable for use in an ELISA assay, together with antibodies specific for isotopes present on the peptides and polypeptides expressed from the aforementioned defined set of genes, suitable reagents for signal detection and amplification and an instruction sheet. In yet another embodiment, the kit comprises antibodies specific for isotopes present on the peptides and polypeptides expressed from the aforementioned defined set of genes, together with reagents suitable for signal detection and amplification using standard immunochemical methods and an instruction sheet.

All the above and other characteristics and advantages of the present invention will be further understood from the following illustrative and non-limitative examples of preferred embodiments thereof.

### BRIEF DESCRIPTION OF THE DRAWINGS

FIG. 1 illustrates the hierarchical clustering, Kaplan-Meier PFS analysis and gene clusters of Ewing sarcoma tumor samples.

a, Illustration of the two sided⁰ clusters dendogram, distinctly defining poor prognosis (1^{st} 8 columns from left to right) vs. good prognosis (6 right-most columns) groups of ES patients and the differentially expressed genes. Each column represents a patient and each row represents a gene.
b, Kaplan-Meier progression free survival analysis presents a significant correlation between poor prognosis vs. good prognosis patients, according to the microarray classification.
c, The 2 major gene clusters and the 6 subclusters, formed on the basis of the similarities of the 818 genes measured over the 14 tumor samples. The 2 gene clusters consist of differentially expressed genes: over-expressed in the poor prognosis group and down-regulated in the good prognosis group, and vice versa.

FIG. 2 graphically illustrates the correlation between expression of the cadherin-11 and the MTA1 genes by microarray analysis and by Real Time PCR.

a, Expression mean log value of cadherin-11 in poor prognosis patients was significantly higher than the expression mean value in good prognosis patients by both analyses.
b, Gene expression pattern in the poor and good prognosis patients, was also significantly correlated by both analyses, for the MTA1 gene.

### DETAILED DESCRIPTION OF PREFERRED EMBODIMENTS

As mentioned, hereinabove, ES is the second most common primary malignant bone tumor in children and adolescents. In spite of advances in multimodal therapy, about 50% of patients eventually relapse, even after 5 years or more. Currently accepted clinical prognostic factors, fail to classify ES patients' risk to relapse at diagnosis.

The recent development of DNA microarrays provides an opportunity to take a genome-wide approach to extend biological insights into all aspects of the study of disease: pathogenesis, disease development, staging, prognosis and treatment response. Gene expression profiling using oligonucleotide high-density arrays has provided an additional tool for elucidating tumor biology as well as the potential for molecular classification of cancer.

In the method of the present invention, oligonucleotide high-density array analysis of material derived from primary tumors is used to identify two distinct gene expression profiles distinguishing ES patients with poor and good prognosis. The results obtained with this method (including the results presented in the Example hereinbelow) indicate the existence of a specific gene expression signature of outcome in ES, already at diagnosis thereby providing a strategy, based upon gene expression patterns, for selecting patients who would benefit from risk adapted improved therapy. The gene expression patterns used in this strategy are based on data sets containing a minimum of 1 significant gene out of the 818 genes to a maximum of 818 genes. Intermediate-sized datasets containing up to 100 genes, 200 genes, 300 genes, 400 genes, 500 genes, 600 genes, 700 genes and 800 genes, may also be usefully defined and used in said selection and prognostic strategy. The present invention also encompasses nucleic acid bearing microarrays for use in the method disclosed herein, as well as kits containing all of the necessary materials and instructions for performing the abovementioned strategy or method, as disclosed and described in more detail hereinbelow.

The details of the aforementioned group of 818 genes for use in accordance with a particularly preferred embodiment of the present invention are listed in Table 1:

TABLE 1

| Gene | Gene Name | Gene Bank ID |
|---|---|---|
| FLII | flightless I homolog (Drosophila) | U80184 |
| PM5 | pM5 protein | X57398 |
| PBEF | pre-B-cell colony-enhancing factor | U02020 |
| KIAA0892 | KIAA0892 protein | AB020699 |
| HSD17B4 | hydroxysteroid (17-beta) dehydrogenase 4 | X87176 |
| IGKC | immunoglobulin kappa constant | X96754 |
| CDC14B | CDC14 cell division cycle 14 homolog B (S. cerevisiae) | AI739548 |
| SLC22A6 | “solute carrier family 22 (organic anion transporter), | AB009698 |
| | member 6” | |
| NRTN | neurturin | U78110 |
| KIAA1096 | KIAA1096 protein | AL096857 |
| IFRD1 | interferon-related developmental regulator 1 | AC005192 |
| KIAA0310 | KIAA0310 gene product | AB002308 |
| ACAA1 | acetyl-Coenzyme A acyltransferase 1 (peroxisomal 3- | X14813 |
| | oxoacyl-Coenzyme A thiolase) | |
| GRN | granulin | AF055008 |
| SH3BGR | SH3 domain binding glutamic acid-rich protein | X93498 |
| MJD | “Machado-Joseph disease (spinocerebellar ataxia 3, | U64820 |
| | olivopontocerebellar ataxia 3, autosomal dominant, | |
| | ataxin 3)” | |
| DKFZP564G2022 | DKFZP564G2022 protein | AL049944 |
| EWSR1 | Ewing sarcoma breakpoint region 1 | X66899 |
| AHCYL1 | S-adenosylhomocysteine hydrolase-like 1 | AI800578 |
| KLRC3 | “killer cell lectin-like receptor subfamily C, member 3” | AJ001685 |
| F2RL1 | coagulation factor II (thrombin) receptor-like 1 | U34038 |
| EIF4G1 | “eukaryotic translation initiation factor 4 gamma, 1” | D12686 |
| | | D26561 |
| TP53BP2 | “tumor protein p53 binding protein, 2” | U58334 |
| TP63 | tumor protein p63 | Y16961 |
| MAN2B1 | “mannosidase, alpha, class 2B, member 1” | U60899 |
| BLCAP | bladder cancer associated protein | AL049288 |
| TAF6 | “TAF6 RNA polymerase II, TATA box binding protein | L25444 |
| | (TBP)-associated factor, 80 kDa” | |
| | H. sapiens hsr1 mRNA (partial) | X66436 |
| STRN3 | “striatin, calmodulin binding protein 3” | U17989 |
| KIAA0914 | KIAA0914 gene product | AB020721 |
| SYNE-2 | synaptic nuclei expressed gene 2 | AL080133 |
| LLGL1 | lethal giant larvae homolog 1 (Drosophila) | X86371 |
| | | M62302 |
| PSMD9 | “proteasome (prosome, macropain) 26S subunit, non- | AB003177 |
| | ATpase, 9” | |
| IL4 | interleukin 4 | M13982 |
| EP400 | E1A binding protein p400 | AI143868 |
| DPAGT1 | dolichyl-phosphate (UDP-N-acetylglucosamine) N- | Z82022 |
| | acetylglucosaminephosphotransferase 1 (GlcNAc-1-P | |
| | transferase) | |
| MKNK1 | MAP kinase-interacting serine/threonine kinase 1 | AB000409 |
| KIAA0356 | KIAA0356 gene product | AB002354 |
| MET | met proto-oncogene (hepatocyte growth factor receptor) | J02958 |
| TPO | thyroid peroxidase | J02969 |
| EGFL5 | “EGF-like-domain, multiple 5” | AB011542 |
| RRS1 | homolog of yeast ribosome biogenesis regulatory protein | D25218 |
| | RRS1 | |
| ARL1 | ADP-ribosylation factor-like 1 | L28997 |
| SDCBP | syndecan binding protein (syntenin) | AF000652 |
| B7 | B7 protein | U72508 |
| SDBCAG84 | serologically defined breast cancer antigen 84 | AF091085 |
| REL | Homo sapiens mRNA; cDNA DKFZp434M162 (from | W72239 |
| | clone DKFZp434M162) | |
| | v-rel reticuloendotheliosis viral oncogene homolog | AA872560 |
| | (avian) | |
| SEMA3F | “sema domain, immunoglobulin domain (Ig), short basic | U38276 |
| | domain, secreted, (semaphorin) 3F” | |
| | | X71346 |
| KLK3 | “kallikrein 3, (prostate specific antigen)” | X07730 |
| F7 | coagulation factor VII (serum prothrombin conversion | M13232 |
| | accelerator) | |
| RBBP2 | retinoblastoma binding protein 2 | S66431 |
| KIAA0020 | KIAA0020 gene product | D13645 |
| GRIN2A | “glutamate receptor, ionotropic, N-methyl D-aspartate | U09002 |
| | 2A” | |
| GART | “phosphoribosylglycinamide formyltransferase, | X54199 |
| | phosphoribosylglycinamide synthetase, | |
| | phosphoribosylaminoimidazole synthetase” | |
| PSMB8 | “proteasome (prosome, macropain) subunit, beta type, 8 | X87344 |
| | (large multifunctional protease 7)” | |
| HTR2A | 5-hydroxytryptamine (serotonin) receptor 2A | AA418537 |
| SURB7 | SRB7 suppressor of RNA polymerase B homolog (yeast) | U52960 |
| MAP3K7IP2 | mitogen-activated protein kinase kinase kinase 7 | AB018276 |
| | interacting protein 2 | |
| MGST3 | microsomal glutathione S-transferase 3 | AF026977 |
| PFDN1 | prefoldin 1 | D45333 |
| U2AF65 | U2 small nuclear ribonucleoprotein auxiliary factor | AI762438 |
| | (65 kD) | |
| KRTHA2 | “keratin, hair, acidic, 2” | X90761 |
| POU4F1 | “POU domain, class 4, transcription factor 1” | L20433 |
| CTSO | cathepsin O | AI810485 |
| MAPK9 | mitogen-activated protein kinase 9 | U09759 |
| ISLR | immunoglobulin superfamily containing leucine-rich | AB003184 |
| | repeat | |
| DKFZP566B183 | DKFZP566B183 protein | AL050272 |
| USP24 | ubiquitin specific protease 24 | AB028980 |
| PBX2 | pre-B-cell leukemia transcription factor 2 | X59842 |
| HT012 | uncharacterized hypothalamus protein HT012 | AI760162 |
| | | X17360 |
| | | HG162-HT3165 |
| HRIHFB2206 | HRIHFB2206 protein | L10379 |
| SYBL1 | synaptobrevin-like 1 | X92396 |
| GRM4 | “glutamate receptor, metabotropic 4” | X80818 |
| ATP5H | “ATP synthase, H+ transporting, mitochondrial F0 | AF087135 |
| | complex, subunit d” | |
| MGC5149 | hypothetical protein MGC5149 | U79260 |
| C20orf188 | chromosome 20 open reading frame 188 | AF055022 |
| ZNF238 | zinc finger protein 238 | U38896 |
| KIAA1030 | KIAA1030 protein | AB028953 |
| PLU-1 | putative DNA/chromatin binding motif | AJ132440 |
| CCT8 | “chaperonin containing TCP1, subunit 8 (theta)” | D13627 |
| XRCC2 | X-ray repair complementing defective repair in Chinese | Y08837 |
| | hamster cells 2 | |
| KIAA0170 | KIAA0170 gene product | AL041663 |
| LPIN2 | lipin 2 | D87436 |
| SULT4A1 | “sulfotransferase family 4A, member 1” | W25958 |
| CDX2 | caudal type homeo box transcription factor 2 | U51096 |
| CFDP1 | craniofacial development protein 1 | D85939 |
| | | HG1155-HT4822 |
| CDK2 | cyclin-dependent kinase 2 | M68520 |
| KIAA0737 | KIAA0737 gene product | AF014837 |
| NTSR2 | neurotensin receptor 2 | Y10148 |
| PRSS15 | “protease, serine, 15” | X76040 |
| UBE2M | “ubiquitin-conjugating enzyme E2M (UBC12 homolog, | AF075599 |
| | yeast)” | |
| NEUROD2 | neurogenic differentiation 2 | AB021742 |
| PCBP3 | poly(rC) binding protein 3 | AL046394 |
| CDK5 | cyclin-dependetent kinase 5 | L04658 |
| UBE3B | ubiquitin protein ligase | AL096740 |
| ALDH9A1 | “aldehyde dehydrogenase 9 family, member A1” | U34252 |
| HCS | cytochrome c | D00265 |
| TUFM | “Tu translation elongation factor, mitochondrial” | S75463 |
| TFCP2 | transcription factor CP2 | U03494 |
| KIAA0963 | KIAA0963 protein | AI760801 |
| SIAH1 | seven in absentia hamolog 1 (Drosophila) | W26406 |
| CRHR2 | corticotropin releasing hormone receptor 2 | AF011406 |
| SLC7A11 | “solute carrier family 7, (cationic amino acid transporter, | AB026891 |
| | y+ system) member 11” | |
| COL6A1 | “collagen, type VI, alpha 1” | AA885106 |
| PTENP1 | “phosphatase and tensin homolog (mutated in multiple | AF019083 |
| | advanced cancers 1), pseudogene 1” | |
| PDAP1 | PDGFA associated protein 1 | U41745 |
| | | U05681 |
| RAD50 | RAD50 homolog (S. cerevisiae) | U63139 |
| | | M13970 |
| LRBA | “LPS-responsive vesicle trafficking, beach and anchor | M83822 |
| | containing” | |
| ARS2 | arsenate resistance protein ARS2 | AI972631 |
| | | AJ002428 |
| ANXA2P1 | annexin A2 pseudogene 1 | M62896 |
| ERCC2 | “excision repair cross-complementing rodent repair | AA079018 |
| | deficiency, complementation group 2 (xeroderma | |
| | pigmentosum D)” | |
| ORC3L | “origin recognition complex, subunit 3-like (yeast)” | AL080116 |
| TNFRSF12 | “tumor necrosis factor receptor superfamily, member 12 | U83598 |
| | (translocating chain-association membrane protein)” | |
| COX6A1 | cytochrome c oxidase subunit VIa polypeptide 1 | AI540925 |
| PRL | prolactin | M29386 |
| PIM1 | pim-1 oncogene | M54915 |
| | Homo sapiens mRNA full length insert cDNA clone | AL109702 |
| | EUROIMAGE 42138 | |
| CCBP2 | chemokine binding protein 2 | U94888 |
| PTS | 6-pyruvoyltetrahydropterin synthase | L76259 |
| GSTA4 | glutathione S-transferase A4 | AF025887 |
| PRSS25 | “protease, serine, 25” | AF020760 |
| SEC14L1 | SEC14-like 1 (S. cerevisiae) | D67029 |
| FGF18 | fibroblast growth factor 18 | AA022949 |
| | | U46194 |
| FLJ20580 | hypothetical protein FLJ20580 | AI862521 |
| DKFZP586B0923 | DKFZP586B0923 protein | AL050190 |
| | Homo sapiens mRNA; cDNA DKFZp434A012 (from | AL096752 |
| | clone DKFZp434A012) | |
| PTK2B | protein tyrosine kinase 2 beta | U43522 |
| RNF13 | ring finger protein 13 | AF037204 |
| ATR | ataxia telangiectasia and Rad3 related | U49844 |
| USP19 | ubiquitin specific protease 19 | AB020698 |
| DDX21 | DEAD/H (Asp-Glu-Ala-Asp/His) box polypeptide 21 | U41387 |
| STK3 | “serine/threonine kinase 3 (STE20 homolog, yeast)” | U26424 |
| MAAT1 | melanoma-associated antigen recognised by cytotoxic T | U19796 |
| | lymphocytes | |
| | | W28193 |
| TMEM1 | transmembrane protein 1 | AB001523 |
| MYB | v-myb myeloblastosis viral oncogene homolog (avian) | M13666 |
| RER1 | similar to S. cerevisiae RER1 | AW044624 |
| RBM9 | RNA binding motif protein 9 | AA402524 |
| DKFZP586A0522 | DKFZP586A0522 protein | AL050159 |
| MVK | mevalonate kinase (mevalonic aciduria) | M88468 |
| CHIT1 | chitinase 1 (chitotriosidase) | U29615 |
| | “Homo sapiens cDNA FLJ32313 fis, clone | AI932613 |
| | PROST2003232, weakly similar to BETA- | |
| | GLUCURONIDASE PRECURSOR (EC 3.2.1.31)” | |
| KIAA1079 | KIAA1079 protein | AI971726 |
| TCFL4 | transcription factor-like 4 | AW005997 |
| UBE2B | ubiquitin-conjugating enzyme E2B (RAD6 homolog) | M74525 |
| HR44 | Hr44 antigen | X91103 |
| CDC5L | CDC5 cell division cycle 5-like (S. pombe) | AB007892 |
| EIF4G1 | “eukaryotic translation initiation factor 4 gamma, 1” | AF104913 |
| GNB1 | “guanine nucleotide binding protein (G protein), beta | X04526 |
| | polypeptide 1” | |
| NRG2 | neuregulin 2 | AA706226 |
| XPNPEP1 | “X-prolyl aminopeptidase (aminopeptidase P) 1, soluble” | X95762 |
| ODC1 | ornithine decarboxylase 1 | X16277 |
| ALMS1 | Alstrom syndrome 1 | R40666 |
| VAPB | VAMP (vesicle-associated membrane protein)- | W27026 |
| | associated protein B and C | |
| UTRN | utrophin (homologous to dystrophin) | X69086 |
| GPR49 | G protein-coupled receptor 49 | AF062006 |
| PPP2R4 | “protein phosphatase 2A, regulatory subunit B′ (PR 53)” | X73478 |
| RABGGTB | “Rab geranylgeranyltransferase, beta subunit” | X98001 |
| AP3S2 | “adaptor-related protein complex 3, sigma 2 subunit” | X99459 |
| KIAA0171 | KIAA0171 gene product | D79993 |
| ABCC8 | “ATP-binding cassette, sub-family C (CFTR/MRP), | L78207 |
| | member 8” | |
| LOC51634 | CGI-79 protein | AL050405 |
| | Homo sapiens clone 24487 mRNA sequence | AF070579 |
| SAH | SA hypertension-associated homolog (rat) | X80062 |
| TCF8 | transcription factor 8 (represses interleukin 2 expression) | U19969 |
| ADCYAP1 | adenylate cyclase activating polypeptide 1 (pituitary) | X60435 |
| DEK | DEK oncogene (DNA binding) | X64229 |
| DBP | D site of albumin promoter (albumin D-box) binding | U48213 |
| | protein | |
| ITGAE | “integrin, alpha E (antigen CD103, human mucosal | L25851 |
| | lymphocyte antigen 1; alpha polypeptide)” | |
| ABCF2 | “ATP-binding cassette, sub-family F (GCN20), member | AJ005016 |
| | 2” | |
| SC5DL | “sterol-C5-desaturase (ERG3 delta-5-desaturase | AB016247 |
| | homolog, fungal)-like” | |
| | | D50525 |
| LGALS9 | “lectin, galactoside-binding, soluble, 9 (galectin 9)” | Z49107 |
| CUL1 | cullin 1 | U58087 |
| GYPE | glycophorin E | X53004 |
| DIAPH2 | diaphanous homolog 2 (Drosophila) | Y15909 |
| PSR | phosphatidylserine receptor | AI950382 |
| LIPA | “lipase A, lysosomal acid, cholesterol esterase (Wolman | X76488 |
| | disease)” | |
| PSMD11 | “proteasome (prosome, macropain) 26S subunit, non- | AB003102 |
| | ATPase, 11” | |
| PSMA3 | “proteasome (prosome, macropain) subunit, alpha type, | D00762 |
| | 3” | |
| VBP1 | von Hippel-Lindau binding protein 1 | U56833 |
| SIX6 | sine oculis homeobox homolog 6 (Drosophila | AJ011785 |
| RBL2 | retinoblastoma-like 2 (p130) | X76061 |
| KCNAB1 | “potassium voltage-gated channel, shaker-related | X83127 |
| | subfamily, beta member 1” | |
| EP300 | E1A binding protein p300 | U01877 |
| ABO | “ABO blood group (transferase A, alpha 1-3-N- | X84746 |
| | acetylgalactosaminyltransferase; transferase B, alpha 1- | |
| | 3-galactosyltransferase)” | |
| GRIK5 | “glutamate receptor, ionotropic, kainate 5” | AA977136 |
| ADPRTL1 | ADP-ribosyltransferase (NAD+; poly (ADP-ribose) | AF057160 |
| | polymerase)-like 1 | |
| HBXIP | hepatitis B virus x interacting protein | AF029890 |
| BHC80 | BRAF35/HDAC2 complex (80 kDa) | W25985 |
| KIAA0436 | putative L-type neutral amino acid transporter | AB007896 |
| MDH2 | “malate dehydrogenase 2, NAD (mitochondrial)” | AF047470 |
| KIAA0630 | KIAA0630 protein | AB014530 |
| IL1RL1 | interleukin 1 receptor-like 1 | D12763 |
| DMTF1 | cyclin D binding myb-like transcription factor 1 | AF052102 |
| MLH1 | “mutL homolog 1, colon cancer, nonpolyposis type 2 (E. coli)” | U07418 |
| GGTLA1 | gamma-glutamyltransferase-like activity 1 | M64099 |
| FHIT | fragile histidine triad gene | U46922 |
| | “ESTs, Weakly similar to I38724 mitochondrial | AI052224 |
| | benzodiazepine receptor - human [H. sapiens]” | |
| ZNF278 | zinc finger protein 278 | AI352450 |
| HLCS | holocarboxylase synthetase (biotin-[proprionyl- | D87328 |
| | Coenzyme A-carboxylase (ATP-hydrolysing)] ligase) | |
| LOC57147 | hypothetical protein LOC57147 | W26641 |
| HTR4 | 5-hydroxytryptamine (serotonin) receptor 4 | Y12505 |
| MORF | monocytic leukemia zinc finger protein-related factor | AB002381 |
| AANAT | arylalkylamine N-acetyltransferase | U40391 |
| MGP | matrix Gla protein | AI953789 |
| | | AB012229 |
| FLJ13052 | NAD kinase | AL031282 |
| VAPB | VAMP (vesicle-associated membrane protein)- | W25933 |
| | associated protein B and C | |
| ENTPD1 | ectonucleoside triphosphate diphosphohydrolase 1 | AJ133133 |
| SDF2 | stromal cell-derived factor 2 | D50645 |
| | | U60269 |
| KIAA0907 | KIAA0907 protein | AB020714 |
| SPRR2C | small proline-rich protein 2C | M21539 |
| DNAJB5 | “DnaJ (Hsp40) homolog, subfamily B, member 5” | AF088982 |
| FMR2 | fragile X mental retardation 2 | U48436 |
| SLC7A8 | “solute carrier family 7 (cationic amino acid transporter, | Y18483 |
| | y+ system), member 8” | |
| E2F5 | “E2F transcription factor 5, p130-binding” | U31556 |
| LSM3 | Lsm3 protein | N98670 |
| FLJ22678 | hypothetical protein FLJ22678 | AA165701 |
| PRKCABP | “protein kinase C, alpha binding protein” | AL049654 |
| DIP2 | disco-interacting protein 2 (Drosophila) homolog | D80006 |
| CEP1 | centrosomal protein 1 | AF083322 |
| PAX6 | “paired box gene 6 (aniridia, keratitis)” | M93650 |
| HLALS | “major histocompatibility complex, class I-like sequence” | AF031469 |
| MPV17 | “MpV17 transgene, murine homolog, glomerulosclerosis” | X76538 |
| | | W29045 |
| KIAA0217 | KIAA0217 protein | D86971 |
| RANBP7 | RAN binding protein 7 | AF098799 |
| UBE4A | “ubiquitination factor E4A (UFD2 homolog, yeast)” | D50916 |
| KIAA0337 | KIAA0337 gene product | AB002335 |
| UPK1A | uroplakin 1A | AF085807 |
| ELAVL2 | “ELAV (embryonic lethal, abnormal vision, Drosophila)- | U29943 |
| | like 2 (HU antigen B)” | |
| PISD | phosphatidylserine decarboxylase | AL050371 |
| ZP3A | zona pellucida glycoprotein 3A (sperm receptor) | X56777 |
| HDAC3 | histone deacetylase 3 | U75697 |
| AD024 | AD024 protein | W28610 |
| PFKFB2 | “6-phosphofructo-2-kinase/fructose-2,6-biphosphatase 2” | AJ005577 |
| RRH | retinal pigment epithelium-derived rhodopsin homolog | AF012270 |
| IGHMBP2 | immunoglobulin mu binding protein 2 | L14754 |
| DSPG3 | dermatan sulfate proteogylcan 3 | U59111 |
| | Homo sapiens mRNA; cDNA DKFZp434M245 (from | W28661 |
| | clone DKFZp434M245) | |
| MAPK9 | mitogen-activated protein kinase 9 | U09759 |
| | | U64871 |
| AMMECR1 | “Alport syndrome, mental retardation, midface | AJ007014 |
| | hypoplasia and elliptocytosis chromosomal region, gene | |
| | 1” | |
| ATP6V1D | “ATPase, H+ transporting, lysosomal 34 kDa, V1 subunit | AA877795 |
| | D” | |
| ANP32A | “acidic (leucine-rich) nuclear phosphoprotein 32 family, | U73477 |
| | member A” | |
| PFAS | phosphoribosylformylglycinamidine synthase (FGAR | AB002359 |
| | amidotransferase) | |
| CPNE3 | copine III | AB014536 |
| KIAA0410 | KIAA0410 gene product | AB007870 |
| SET | SET translocation (myeloid leukemia-associated) | M93651 |
| CSTF2 | “cleavage stimulation factor, 3′ pre-RNA, subunit 2, | M85085 |
| | 64 kDa” | |
| ASNA1 | “arsA arsenite transporter, ATP-binding, homolog 1 | AF047469 |
| | (bacterial)” | |
| SLC2A1 | “solute carrier family 2 (facilitated glucose transporter), | K03195 |
| | member 1” | |
| C8orf1 | chromosome 8 open reading frame 1 | AI738702 |
| | Homo sapiens mRNA; cDNA DKFZp586K2322 (from | AL080113 |
| | clone DKFZp586K2322) | |
| TM9SF1 | transmembrane 9 superfamily member 1 | U94831 |
| NDP | Norrie disease (pseudoglioma) | X65724 |
| YWHAE | “tyrosine 3-monooxygenase/tryptophan 5- | U54778 |
| | monooxygenase activation protein, epsilon polypeptide” | |
| KCNJ6 | “potassium inwardly-rectifying channel, subfamily J, | U52153 |
| | member 6” | |
| | | X03453 |
| RFPL3 | ret finger protein-like 3 | AJ010232 |
| HCFC1 | host cell factor C1 (VP16-accessory protein) | U52112 |
| SLC12A4 | “solute carrier family 12 (potassium/chloride | AF054506 |
| | transporters), member 4” | |
| T | “T, brachyury homolog (mouse)” | AJ001699 |
| ZNF174 | zinc finger protein 174 | U31248 |
| TRAP100 | thyroid hormone receptor-associated protein (100 kDa) | D50920 |
| HTR6 | 5-hydroxytryptamine (serotonin) receptor 6 | L41147 |
| NASP | nuclear autoantigenic sperm protein (histone-binding) | M97856 |
| COMT | catechol-O-methyltransferase | M58525 |
| AXL | AXL receptor tyrosine kinase | M76125 |
| NME1 | “non-metastatic cells 1, protein (NM23A) expressed in” | X73066 |
| | | M10098 |
| LOC51055 | unknown | U88048 |
| CREM | cAMP responsive element modulator | S68271 |
| MEF-2 | myelin gene expression factor 2 | W28567 |
| PCBP1 | poly(rC) binding protein 1 | Z29505 |
| GNG5 | “guanine nucleotide binding protein (G protein), gamma | AI541042 |
| | 5” | |
| CNNM2 | cyclin M2 | AI827730 |
| NCSTN | nicastrin | D87442 |
| ICOS | inducible T-cell co-stimulator | AB023135 |
| TK2 | “thymidine kinase 2, mitochondrial” | U80628 |
| LTK | leukocyte tyrosine kinase | X52213 |
| BRD2 | bromodomain containing 2 | D42040 |
| SMAP | skeletal muscle abundant protein | AF016270 |
| | Homo sapiens retinoic acid-inducible endogenous | M64936 |
| | retroviral DNA | |
| MYO1C | myosin IC | X98507 |
| IMAGE145052 | small acidic protein | AI346580 |
| | “AML1 = AML1 {alternatively spliced, exons 5 and b} | S76346 |
| | [human, mRNA Partial, 284 nt]” | |
| IKKE | IKK-related kinase epsilon; inducible IkappaB kinase | D63485 |
| LU | Lutheran blood group (Auberger b antigen included) | X80026 |
| KIAA0828 | KIAA0828 protein | AB020635 |
| SLC30A3 | “solute carrier family 30 (zinc transporter), member 3” | U76010 |
| IL13RA1 | “interleukin 13 receptor, alpha 1” | Y10659 |
| C22orf4 | chromosome 22 open reading frame 4 | AL096779 |
| BCL11A | B-cell CLL/lymphoma 11A (zinc finger protein) | W27619 |
| HIPK3 | homeodomain interacting protein kinase 3 | AI523538 |
| ACVR1B | “activin A receptor, type IB” | Z22536 |
| UBA2 | SUMO-1 activating enzyme subunit 2 | AL041443 |
| THRA | “thyroid hormone receptor, alpha (erythroblastic | X55005 |
| | leukemia viral (v-erb-a) oncogene homolog, avian)” | |
| NCOA2 | nuclear receptor coactivator 2 | AI040324 |
| IRF2 | interferon regulatory factor 2 | X15949 |
| | | L38424 |
| GNAS | GNAS complex locus | X04409 |
| TM4SF6 | transmembrane 4 superfamily member 6 | AF043906 |
| ZK1 | Kruppel-type zinc finger (C2H2) | AB011414 |
| ARPC5 | “actin related protein 2/3 complex, subunit 5, 16 kDa” | AF006088 |
| PEX7 | peroxisomal biogenesis factor 7 | U88871 |
| FMR1 | fragile X mental retardation 1 | X69962 |
| ZP2 | zona pellucida glycoprotein 2 (sperm receptor) | M90366 |
| OR7E126P | “olfactory receptor, family 7, subfamily A, member 126 | AF065854 |
| | pseudogene” | |
| HSF4 | heat shock transcription factor 4 | D87673 |
| | | HG2702-HT2798 |
| UBE2G1 | “ubiquitin-conjugating enzyme E2G 1 (UBC7 homolog, | D78514 |
| | C. elegans)” | |
| GRLF1 | glucocorticoid receptor DNA binding factor 1 | AI670100 |
| SSFA2 | sperm specific antigen 2 | M61199 |
| JIK | STE20-like kinase | W28742 |
| PPP3CC | “protein phosphatase 3 (formerly 2B), catalytic subunit, | AI762547 |
| | gamma isoform (calcineurin A gamma)” | |
| AHCYL1 | S-adenosylhomocysteine hydrolase-like 1 | AI800578 |
| PRCP | prolylcarboxypeptidase (angiotensinase C) | L13977 |
| NR2C1 | “nuclear receptor subfamily 2, group C, member 1” | M29960 |
| FUS | “fusion, derived from t(12; 16) malignant liposarcoma” | S62140 |
| ZNF273 | zinc finger protein 273 | X78932 |
| MYST1 | MYST histone acetyltransferase 1 | AI417075 |
| NQO1 | “NAD(P)H dehydrogenase, quinone 1” | M81600 |
| ADAM15 | a disintegrin and metalloproteinase domain 15 | U41767 |
| | (metargidin) | |
| CRYAB | “crystallin, alpha B” | AL038340 |
| DKFZp566D133 | DKFZp566D133 protein | AL050050 |
| MAPRE1 | “microtubule-associated protein, RP/EB family, member | U24166 |
| | 1” | |
| TGFB1 | “transforming growth factor, beta 1 (Camurati- | X02812 |
| | Engelmann disease)” | |
| ZNF189 | zinc finger protein 189 | AF025770 |
| ATP1B3 | “ATPase, Na+/K+ transporting, beta 3 polypeptide” | U51478 |
| TG737 | “Probe hTg737 (polycystic kidney disease, autosomal | U20362 |
| | recessive, in)” | |
| FST | follistatin | M19481 |
| DKFZP564O0423 | DKFZP564O0423 protein | AL080120 |
| MAGEA4 | “melanoma antigen, family A, 4” | U10688 |
| POU6F1 | “POU domain class 6, transcription factor 1” | Z21966 |
| FLJ20986 | hypothetical protein FLJ20986 | Z24724 |
| LOC90586 | amine oxidase pseudogene | AF047485 |
| MIPEP | mitochondrial intermediate peptidase | U80034 |
| | Homo sapiens clone 24507 mRNA sequence | AF052148 |
| | Homo sapiens mRNA; cDNA DKFZp667O1814 (from | W26677 |
| | clone DKFZp667O1814) | |
| HTR1E | 5-hydroxytryptamine (serotonin) receptor 1E | M91467 |
| DKFZP564L0862 | DKFZP564L0862 protein | AL080091 |
| HRB2 | HIV-1 rev binding protein 2 | U00943 |
| REA | repressor of estrogen receptor activity | U72511 |
| DOK1 | “docking protein 1, 62 kDa (downstream of tyrosine | U70987 |
| | kinase 1)” | |
| KIAA0710 | KIAA0710 gene product | AB014610 |
| PRNP | “prion protein (p27-30) (Creutzfeld-Jakob disease, | U29185 |
| | Gerstmann-Strausler-Scheinker syndrome, fatal familial | |
| | insomnia)” | |
| PTK7 | PTK7 protein tyrosine kinase 7 | U33635 |
| KIAA0426 | KIAA0426 gene product | AB007886 |
| | “Phosphoglycerate kinase {alternatively spliced} [human, | S81916 |
| | phosphoglycerate kinase deficient patient with episodes | |
| | of muscl, mRNA Partial Mutant, 307 nt]” | |
| NEDD4 | “neural precursor cell expressed, developmentally down- | D42055 |
| | regulated 4” | |
| CSH2 | chorionic somatomammotropin hormone 2 | AA151971 |
| ARF4 | ADP-ribosylation factor 4 | M36341 |
| CD34 | CD34 antigen | M81945 |
| KIAA0092 | KIAA0092 gene product | D42054 |
| DKFZp434G2311 | hypothetical protein DKFZp434G2311 | W22289 |
| GYPB | glycophorin B (includes Ss blood group) | U05255 |
| TIC | SEC7 homolog | U63127 |
| | | X61072 |
| KIAA0552 | KIAA0552 gene product | AB011124 |
| KIAA0970 | KIAA0970 protein | AB023187 |
| SLC18A1 | “solute carrier family 18 (vesicular monoamine), member | U39905 |
| | 1” | |
| | | D86096 |
| S100A5 | S100 calcium binding protein A5 | Z18954 |
| EFNA3 | ephrin-A3 | U14187 |
| NM23-H6 | nucleoside diphosphate kinase type 6 (inhibitor of p53- | AF051941 |
| | induced apoptosis-alpha) | |
| NXF1 | nuclear RNA export factor 1 | AJ132712 |
| SLC4A8 | “solute carrier family 4, sodium bicarbonate | AB018282 |
| | cotransporter, member 8” | |
| IGHM | immunoglobulin heavy constant mu | AF015128 |
| EEF1A1 | eukaryotic translation elongation factor 1 alpha 1 | W28170 |
| | Homo sapiens clone 24468 mRNA sequence | AF070623 |
| USP9X | “ubiquitin specific protease 9, X chromosome (fat facets- | X98296 |
| | like Drosophila)” | |
| DYRK2 | dual-specificity tyrosine-(Y)-phosphorylation regulated | Y09216 |
| | kinase 2 | |
| LBP | lipopolysaccharide binding protein | AF013512 |
| POH1 | 26S proteasome-associated pad1 homolog | U86782 |
| KIAA0211 | KIAA0214 gene product | D86966 |
| PXR1 | peroxisome receptor 1 | Z48054 |
| | | HG2689-HT2785 |
| TAF4 | “TAF4 RNA polymerase II, TATA box binding protein | U75308 |
| | (TBP)-associated factor, 135 kDa” | |
| ZNF313 | zinc finger protein 313 | AL031685 |
| PPAP2A | phosphatidic acid phosphatase type 2A | AF014402 |
| FLJ20323 | hypothetical protein FLJ20323 | AC004982 |
| TCP1 | t-complex 1 | X52882 |
| NR2F1 | “nuclear receptor subfamily 2, group F, member 1” | X16155 |
| MAG | myelin associated glycoprotein | M29273 |
| | | J04423 |
| ELAC2 | elaC homolog 2 (E. coli) | AA522537 |
| MAPKAPK2 | mitogen-activated protein kinase-activated protein kinase 2 | U12779 |
| SMAP | skeletal muscle abundant protein | X87613 |
| ZNF263 | zinc finger protein 263 | D88827 |
| DDX27 | DEAD/H (Asp-Glu-Ala-Asp/His) box polypeptide 27 | W25911 |
| HSA6591 | nucleolar cysteine-rich protein | AJ006591 |
| MAGOH | “mago-nashi homolog, proliferation-associated | AF035940 |
| | (Drosophila)” | |
| | | Y16788 |
| KRT2A | keratin 2A (epidermal ichthyosis bullosa of Siemens) | AF019084 |
| RALY | “RNA binding protein (autoantigenic, hnRNP-associated | L38696 |
| | with lethal yellow)” | |
| C11orf9 | chromosome 11 open reading frame 9 | AB023171 |
| XPO1 | “exportin 1 (CRM1 homolog, yeast)” | Y08614 |
| H2BFC | “H2B histone family, member C” | AL009179 |
| SETDB1 | “SET domain, bifurcated 1” | D31891 |
| SEC63L | SEC63 protein | AJ011779 |
| MGC8721 | hypothetical protein MGC8721 | W26659 |
| RPP40 | “ribonuclease P, 40 kD subunit” | U94317 |
| GAPD | glyceraldehyde-3-phosphate dehydrogenase | M33197 |
| KIAA0467 | KIAA0467 protein | AB007936 |
| KCNMB1 | “potassium large conductance calcium-activated | U25138 |
| | channel, subfamily M, beta member 1” | |
| PML | promyelocytic leukemia | M79463 |
| B2M | beta-2-microglobulin | S82297 |
| UROS | uroporphyrinogen III synthase (congenital erythropoietic | J03824 |
| | porphyria) | |
| PDE4A | “phosphodiesterase 4A, cAMP-specific | L20965 |
| | (phosphodiesterase E2 dunce homolog, Drosophila)” | |
| | | M59830 |
| NUP155 | nucleoporin 155 kDa | AB018334 |
| HRMT1L1 | HMT1 hnRNP methyltransferase-like 1 (S. cerevisiae) | X99209 |
| BTN3A2 | “butyrophilin, subfamily 3, member A2” | U97502 |
| TRAP100 | thyroid hormone receptor-associated protein (100 kDa) | W29091 |
| PRKCD | “protein kinase C, delta” | D10495 |
| OAZ2 | ornithine decarboxylase antizyme 2 | AF057297 |
| ADRBK1 | “adrenergic, beta, receptor kinase 1” | U08438 |
| | “Homo sapiens cDNA FLJ30824 fis, clone | H12054 |
| | FEBRA2001698” | |
| GTF2H4 | “general transcription factor IIH, polypeptide 4, 52 kDa” | Y07595 |
| LGALS9 | “lectin, galactoside-binding, soluble, 9 (galectin 9)” | AB006782 |
| ACTB | “actin, beta” | X00351 |
| TMSB4Y | “thymosin, beta 4, Y chromosome” | AF000989 |
| GTF3C2 | “general transcription factor IIIC, polypeptide 2, beta | D13636 |
| | 110 kDa” | |
| C9orf3 | chromosome 9 open reading frame 3 | AF043897 |
| NSEP1 | nuclease sensitive element binding protein 1 | M85234 |
| TNP1 | transition protein 1 (during histone to protamine | X07948 |
| | replacement) | |
| | | D10995 |
| HEXA | hexosaminidase A (alpha polypeptide) | M16424 |
| CCNF | cyclin F | Z36714 |
| | | AL034450 |
| SIP | Siah-interacting protein | AL035305 |
| | | X81832 |
| HLA-F | “major histocompatibility complex, class I, F” | AL022723 |
| DKFZP434D1335 | DKFZP434D1335 protein | AI920820 |
| RNASEH1 | ribonuclease H1 | AF039652 |
| | “Homo sapiens cDNA: FLJ23482 fis, clone KAIA03142” | U55980 |
| KIAA0877 | KIAA0877 protein | AB020684 |
| CLTB | “clathrin, light polypeptide (Lcb)” | X81637 |
| HSPA8 | heat shock 70 kDa protein 8 | Y00371 |
| CTNNA1 | “catenin (cadherin-associated protein), alpha 1 (102 kDa” | U03100 |
| | | W27906 |
| EIF4A2 | “eukaryotic translation initiation factor 4A, isoform 2” | D30655 |
| H2BFN | “H2B histone family, member N” | Z98744 |
| KIAA0514 | KIAA0514 gene product | AB011086 |
| PRPS1 | phosphoribosyl pyrophosphate synthetase 1 | D00860 |
| PAX8 | paired box gene 8 | X69699 |
| | | U10689 |
| B4GALT4 | “UDP-Gal:betaGlcNAc beta 1,4-galactosyltransferase, | AF038662 |
| | polypeptide 4” | |
| | Homo sapiens clone 23821 mRNA sequence | AF038194 |
| PAFAH1B1 | “platelet-activating factor acetylhydrolase, isoform Ib, | L13385 |
| | alpha subunit 45 kDa” | |
| IFNA10 | “interferon, alpha 10” | V00551 |
| ABCB10 | “ATP-binding cassette, sub-family B (MDR/TAP), | U18237 |
| | member 10” | |
| CASP10 | “caspase 10, apoptosis-related cysteine protease” | U60519 |
| PFKM | “phosphofructokinase, muscle” | U24183 |
| RCN2 | “reticulocalbin 2, EF-hand calcium binding domain” | X78669 |
| PPP3CB | “protein phosphatase 3 (formerly 2B), catalytic subunit, | M29550 |
| | beta isoform (calcineurin A beta)” | |
| H6PD | hexose-6-phosphate dehydrogenase (glucose 1- | AJ012590 |
| | dehydrogenase) | |
| PTPRA | “protein tyrosine phosphatase, receptor type, A” | M34668 |
| FUT7 | “fucosyltransferase 7 (alpha (1,3) fucosyltransferase)” | AB012668 |
| PFKP | “phosphofructokinase, platelet” | D25328 |
| MAGEA9 | “melanoma antigen, family A, 9” | U10694 |
| SDFR1 | stromal cell derived factor receptor 1 | AF035287 |
| CAV2 | caveolin 2 | AF035752 |
| ERCC5 | “excision repair cross-complementing rodent repair | L20046 |
| | deficiency, complementation group 5 (xeroderma | |
| | pigmentosum, complementation group G (Cockayne | |
| | syndrome))” | |
| MLN | motilin | X15393 |
| PTK2 | PTK2 protein tyrosine kinase 2 | L13616 |
| P84 | nuclear matrix protein p84 | L36529 |
| OS4 | conserved gene amplified in osteosarcoma | AF000152 |
| ITPR2 | “inositol 1,4,5-triphosphate receptor, type 2” | D26350 |
| POU6F1 | “POU domain, class 6, transcription factor 1” | Z21966 |
| GATA2 | GATA binding protein 2 | M77810 |
| SFRS7 | “splicing factor, arginine/serine-rich 7, 35 kDa” | L41887 |
| FBXO21 | F-box only protein 21 | AB020682 |
| AGM1 | N-acetylglucosamine-phosphate mutase | AA001791 |
| UGT2B15 | “UDP glycosyltransferase 2 family, polypeptide B15” | U06641 |
| SGNE1 | “secretory granule, neuroendocrine protein 1 (7B2 | Y00757 |
| | protein)” | |
| CHP | calcium binding protein P22 | U61538 |
| PDCD10 | programmed cell death 10 | AF022385 |
| FLJ21432 | hypothetical protein FLJ21432 | W26655 |
| KIAA0692 | KIAA0692 protein | AI924382 |
| HNRPH3 | heterogeneous nuclear ribonucleoprotein H3 (2H9) | AF052131 |
| OCRL | oculocerebrorenal syndrome of Lowe | U57627 |
| ESR2 | estrogen receptor 2 (ER beta) | X99101 |
| | | HG1111-HT1111 |
| | Homo sapiens mRNA; cDNA DKFZp586I1319 (from | AL050106 |
| | clone DKFZp586I1319) | |
| SIM2 | single-minded homolog 2 (Drosophila) | U80457 |
| DCTN1 | “dynactin 1 (p150, glued homolog, Drosophila)” | AF086947 |
| MGC9651 | hypothetical protein MGC9651 | W21884 |
| SFRS3 | “splicing factor, arginine/serine-rich 3” | AF038250 |
| ZNF10 | zinc finger protein 10 (KOX 1) | X52332 |
| AP2A2 | “adaptor-related protein complex 2, alpha 2 subunit” | AB020706 |
| FLJ10618 | hypothetical protein FLJ10618 | AL049246 |
| TTTY15 | “testis-specific transcript, Y-linked 15” | AL080135 |
| ID1 | “inhibitor of DNA binding 1, dominant negative helix-loop- | X77956 |
| | helix protein” | |
| DAG1 | dystroglycan 1 (dystrophin-associated glycoprotein 1) | L19711 |
| ZNF175 | zinc finger protein 175 | D50419 |
| | | W26472 |
| RAB2 | “RAB2, member RAS oncogene family” | M28213 |
| ENPP4 | ectonucleotide pyrophosphatase/phosphodiesterase 4 | AB020686 |
| | (putative function) | |
| RHBDL | “rhomboid, veinlet-like 1 (Drosophila)” | Y17108 |
| KIAA0648 | KIAA0648 protein | AB014548 |
| UCHL3 | ubiquitin carboxyl-terminal esterase L3 (ubiquitin | AA746355 |
| | thiolesterase) | |
| LOC51035 | ORF | M68864 |
| ITGB2 | “integrin, beta 2 (antigen CD18 (p95), lymphocyte | M15395 |
| | function-associated antigen 1; macrophage antigen 1 | |
| | (mac-1) beta subunit)” | |
| PPP2R5C | “protein phosphatase 2, regulatory subunit B (B56), | Z69030 |
| | gamma isoform” | |
| MIR16 | membrane interacting protein of RGS16 | AC003108 |
| HSPCB | “heat shock 90 kDa protein 1, beta” | M16660 |
| ATP6V1A1 | “ATPase, H+ transporting, lysosomal 70 kDa, V1 subunit | AA056747 |
| | A, isoform 1” | |
| CETN3 | “centrin, EF-hand protein, 3 (CDC31 homolog, yeast)” | AI056696 |
| PRDX3 | peroxiredoxin 3 | D49396 |
| LOC129080 | putative emu1 | AL031186 |
| P2RX5 | “purinergic receptor P2X, ligand-gated ion channel, 5” | U49395 |
| HUMPPA | paraneoplastic antigen | L02867 |
| | | HG2530-HT2626 |
| SCAP | SREBP CLEAVAGE-ACTIVATING PROTEIN | D83782 |
| MD-1 | “MD-1, RP105-associated” | AB020499 |
| CDC6 | CDC6 cell division cycle 6 homolog (S. cerevisiae) | U77949 |
| BRAP | BRCA1 associated protein | AL042733 |
| CAMK2G | calcium/calmodulin-dependent protein kinase (CaM | U66063 |
| | kinase) II gamma | |
| MLCB | “myosin, light polypeptide, regulatory, non-sarcomeric | X54304 |
| | (20 kD)” | |
| OPA1 | optic atrophy 1 (autosomal dominant) | AB011139 |
| HSPC111 | hypothetical protein HSPC111 | AI553745 |
| STK39 | “serine threonine kinase 39 (STE20/SPS1 homolog, | AF099989 |
| | yeast)” | |
| YME1L1 | YME1-like 1 (S. cerevisiae) | AJ132637 |
| H1F2 | “H1 histone family, member 2” | AI189287 |
| MLANA | melan-A | U06452 |
| PSMD9 | “proteasome (prosome, macropain) 26S subunit, non- | AI347155 |
| | ATPase, 9” | |
| LARGE | like-glycosyltransferase | AJ007583 |
| CREB3 | cAMP responsive element binding protein 3 (luman) | U88528 |
| MRPS14 | mitochondrial ribosomal protein S14 | AL049705 |
| TM4SF5 | transmembrane 4 superfamily member 5 | AF027204 |
| SIT | SHP2 interacting transmembrane adaptor | AJ010059 |
| | | Z48950 |
| EPB49 | erythrocyte membrane protein band 4.9 (dematin) | U28389 |
| TCN2 | transcobalamin II; macrocytic anemia | L02648 |
| OIP2 | Opa-Interacting protein 2 | AL050353 |
| ALAS2 | “aminolevulinate, delta-, synthase 2 | X60364 |
| | (sideroblastic/hypochromic anemia)” | |
| CHC1 | chromosome condensation 1 | X12654 |
| GMPS | guanine monphosphate synthetase | U10860 |
| SLC25A14 | “solute carrier family 25 (mitochondrial carrier, brain), | AF078544 |
| | member 14” | |
| HNRPM | heterogeneous nuclear ribonucleoprotein M | L03532 |
| PDZ-GEF1 | PDZ domain containing guanine nucleotide exchange | AB002311 |
| | factor(GEF)1 | |
| UBE2N | “ubiquitin-conjugating enzyme E2N (UBC13 homolog, | D83004 |
| | yeast)” | |
| | “ESTs, Moderately similar to hypothetical protein | W28230 |
| | FLJ20489 [Homo sapiens] [H. sapiens]” | |
| NEDD5 | “neural precursor cell expressed, developmentally down- | M11717 |
| | regulated 5” | D63878 |
| | | J04423 |
| CDH2 | “cadherin 2, type 1, N-cadherin (neuronal)” | M34064 |
| PP35 | protein similar to E. coli yhdg and R. capsulatus nifR3 | U62767 |
| | Homo sapiens mRNA; cDNA DKFZp686N1377 (from | S63912 |
| | clone DKFZp686N1377) | |
| | “Homo sapiens cDNA FLJ13555 fis, clone | AL080210 |
| | PLACE1007677” | |
| | | M33764 |
| RELN | reelin | U79716 |
| PPP1R12A | “protein phosphatase 1, regulatory (inhibitor) subunit | D87930 |
| | 12A” | |
| SLC9A6 | “solute carrier family 9 (sodium/hydrogen exchanger), | AF030409 |
| | isoform 6” | |
| NRXN1 | neurexin 1 | AB011150 |
| 76P | gamma tubulin ring complex protein (76p gene) | W28255 |
| DKFZp564B0769 | SR rich protein | AL080186 |
| ADPRT | ADP-ribosyltransferase (NAD+; poly (ADP-ribose) | J03473 |
| | polymerase) | |
| SRPX | “sushi-repeat-containing protein, X chromosome” | U61374 |
| SAS10 | disrupter of silencing 10 | AI126004 |
| GNAS | GNAS complex locus | X04409 |
| | | X57152 |
| MID2 | midline 2 | AL034399 |
| U5-100K | “prp28, U5 snRNP 100 kd protein” | AF026402 |
| PTPRD | “protein tyrosine phosphatase, receptor type, D” | AA843737 |
| SPTB | “spectrin, beta, erythrocytic (includes spherocytosis, | J05500 |
| | clinical type I)” | |
| CDK6 | cyclin-dependent kinase 6 | AI738463 |
| DPYSL4 | dihydropyrimidinase-like 4 | AB006713 |
| DKFZP566F0546 | DKFZP566F0546 protein | AI671905 |
| CCT2 | “chaperonin containing TCP1, subunit 2 (beta)” | AF026166 |
| PROL2 | proline rich 2 | U03105 |
| | | D00591 |
| | | M13929 |
| DR1 | “down-regulator of transcription 1, TBP-binding (negative | M97388 |
| | cofactor 2)” | |
| | | L00049 |
| MTHFR | “5,10-methylenetetrahydrofolate reductase (NADPH)” | AJ237672 |
| SIMRP7 | multidrug resistance-associated protein 7 | AI004207 |
| CDH11 | “cadherin 11, type 2, OB-cadherin (osteoblast)” | D21255 |
| FLJ11198 | hypothetical protein FLJ11198 | U66685 |
| ATRX | “alpha thalassemia/mental retardation syndrome X-linked | U72936 |
| | (RAD54 homolog, S. cerevisiae)” | |
| BRCA1 | “breast cancer 1, early onset” | U64805 |
| MLLT4 | “myeloid/lymphoid or mixed-lineage leukemia (trithorax | AB011399 |
| | homolog, Drosophila); translocated to, 4” | |
| COX11 | “COX11 homolog, cytochrome c oxidase assembly | U79270 |
| | protein (yeast)” | |
| TCEA1 | “transcrption elongation factor A (SII), 1” | M81601 |
| TEGT | testis enhanced gene transcript (BAX inhibitor 1) | X75861 |
| RPL9 | ribosomal protein L9 | U09953 |
| CDK5R1 | “cyclin-dependent kinase 5, regulatory subunit 1 (p35)” | X80343 |
| | | HG4518-HT4921 |
| SOS2 | son of sevenless homolog 2 (Drosophila) | L13858 |
| EPHB2 | EphB2 | AF025304 |
| | | Z97054 |
| KIAA0185 | KIAA0185 protein | D80007 |
| MYC | v-myc myelocomatosis viral oncogene homolog (avian) | V00568 |
| KCNK3 | “potassium channel, subfamily K, member 3” | AF006823 |
| HSPA9B | heat shock 70 kDa protein 9B (mortalin-2) | L15189 |
| AIF1 | allograft inflammatory factor 1 | Y14768 |
| PMS2L6 | postmeiotic segregation increased 2-like 6 | AI341574 |
| DMWD | dystrophia myotonica-containing WD repeat motif | L19267 |
| GMPR | guanosine monophosphate reductase | M24470 |
| RTP801 | HIF-1 responsive RTP801 | M10098 |
| MMP11 | matrix metalloproteinase 11 (stromelysin 3) | AA522530 |
| | | X57766 |
| KIAA1067 | KIAA1067 protein | AB028990 |
| ADAM19 | a disintegrin and metalloproteinase domain 19 (meltrin | AL049415 |
| | beta) | |
| | Homo sapiens mRNA; cDNA DKFZp586F2224 (from | AI655015 |
| | clone DKFZp586F2224) | |
| C1orf16 | chromosome 1 open reading frame 16 | D87437 |
| GP1BA | “glycoprotein Ib (platelet), alpha polypeptide” | J02940 |
| SDHB | “succinate dehydrogenase complex, subunit B, iron | U17886 |
| | sulfur (Ip)” | |
| NTRK2 | “neurotrophic tyrosine kinase, receptor, type 2” | U12140 |
| KIAA0110 | gene predicted from cDNA with a complete coding | D14811 |
| | sequence | |
| MAP3K7 | mitgen-activated protein kinase kinase kinase 7 | AB009356 |
| MGC5466 | hypothetical protein MGC5466 | U90904 |
| PPM1A | “protein phosphatase 1A (formerly 2C), magnesium- | S87759 |
| | dependent, alpha isoform” | |
| | | K01383 |
| KIAA0677 | KIAA0677 gene product | AB014577 |
| HNRPA2B1 | heterogeneous nuclear ribonucleoprotein A2/B1 | M29065 |
| DKFZP434J046 | DKFZP434J046 protein | AC004144 |
| MAN1A1 | “mannosidase, alpha, class 1A, member 1” | X74837 |
| KIAA0455 | KIAA0455 gene product | AB007924 |
| NUP160 | nucleoporin 160 kDa | D83781 |
| NMT1 | N-myristoyltransferase 1 | M86707 |
| PIP5K1C | “phosphatidylinositol-4-phosphate 5-kinase, type I, | AB011161 |
| | gamma” | |
| GTF2H3 | “general transcription factor IIH, polypeptide 3, 34 kDa” | Z30093 |
| DCN | decorin | M14219 |
| | “Human small proline rich protein (sprII) mRNA, clone | M21302 |
| | 174N” | |
| POLR2B | “polymerase (RNA) II (DNA directed) polypetide B, | X63563 |
| | 140 kDa” | |
| | | J04988 |
| AHSG | alpha-2-HS-glycoprotein | M16961 |
| STAM | signal transducing adaptor molecule (SH3 domain and | U43899 |
| | ITAM motif) 1 | |
| SCAM-1 | vinexin beta (SH3-containing adaptor molecule-1) | AF037261 |
| RAF1 | v-raf-1 murine leukemia viral oncogene homolog 1 | X06409 |
| KIAA0964 | KIAA0964 protein | AB023181 |
| SPARCL1 | “SPARC-like 1 (mast9, hevin)” | X86693 |
| PGRMC1 | progesterone receptor membrane component 1 | Y12711 |
| COPS5 | COP9 constitutive photomorphogenic homolog subunit 5 | U65928 |
| | (Arabidopsis) | |
| MGC2650 | hypothetical protein MGC2650 | AI885381 |
| CYP11A | “cytochrome P450, subfamily XIA (cholesterol side chain | M14565 |
| | cleavage)” | |
| CPB2 | “carboxypeptidase B2 (plasma, carboxypeptidase U)” | M75106 |
| NRG1 | neuregulin 1 | L41827 |
| GTF2F2 | “general transcription factor IIF, polypeptide 2, 30 kDa” | X16901 |
| UCP2 | “uncoupling protein 2 (mitochondrial, proton carrier)” | U94592 |
| BM036 | uncharacterized bone marrow protein BM036 | AI057607 |
| HLA-G | “HLA-G histocompatibility antigen, class I, G” | M90683 |
| SS18L1 | synovial sarcoma translocation gene on chromosome | AB014593 |
| | 18-like 1 | |
| DKFZP547E1010 | DKFZP547E1010 protein | AL050260 |
| PARG | poly (ADP-ribose) glycohydrolase | AF005043 |
| RPS15A | ribosomal protein S15a | W52024 |
| CREBL2 | cAMP responsive element binding protein-like 2 | AF039081 |
| HSD17B3 | hydroxysteroid (17-beta) dehydrogenase 3 | U05659 |
| | Homo sapiens clone 23718 mRNA sequence | AF052138 |
| | | HG2465-HT4871 |
| IDI1 | isopentenyl-diphosphate delta isomerase | X17025 |
| CBX3 | “chromobox homolog 3 (HP1 gamma homolog, | AA648295 |
| | Drosophila)” | |
| PAI-RBP1 | PAI-1 mRNA-binding protein | AL080119 |
| SFPQ | splicing factor proline/glutamine rich (polypyrimidine tract | W27050 |
| | binding protein associated) | |
| AMACR | alpha-methylacyl-CoA racemase | AJ130733 |
| KIAA1045 | KIAA1045 protein | AB028968 |
| HNRPH2 | heterogeneous nuclear ribonucleoprotein H2 (H′) | U01923 |
| KIAA0537 | KIAA0537 gene product | AB011109 |
| | | X55503 |
| MLLT2 | “myeloid/lymphoid or mixed-lineage leukemia (trithorax | L13773 |
| | homolog, Drosophila); translocated to, 2” | |
| ELAVL3 | “ELAV (embryonic lethal, abnormal vision, Drosophila)- | D26158 |
| | like 3 (Hu antigen C)” | |
| ING1L | “inhibitor of growth family, member 1-like” | AI186701 |
| PPP4R1 | “protein phosphatase 4, regulatory subunit 1” | U79267 |
| ACTB | “actin, beta” | X63432 |
| FBXO9 | F-box only protein 9 | AL031178 |
| LYPLA1 | lysophospholipase I | AF081281 |
| POLR3F | “polymerase (RNA) III (DNA directed) polypeptide F, 39 kDa” | U93869 |
| MCLC | Mid-1-related chloride channel 1 | AB018304 |
| PPIE | peptidylprolyl isomerase E (cyclophilin E) | AF042386 |
| PAICS | “phosphoribosylaminoimidazole carboxylase, | X53793 |
| | phosphoribosylaminoimidazole succinocarboxamide | |
| | synthetase” | |
| IFNGR2 | interferon gamma receptor 2 (interferon gamma | U05875 |
| | transducer 1) | |
| PITPNM | “phosphatidylinositol transfer protein, membrane- | X98654 |
| | associated” | |
| | | X03453 |
| KIAA0435 | KIAA0435 gene product | AB007895 |
| TAZ | “tafazzin (cardiomyopathy, dilated 3A (X-linked); | X92762 |
| | endocardial fibroelastosis 2; Barth syndrome)” | |
| ATP6V1H | “ATPase, H+ transporting, lysosomal 50/57 kDa, V1 | AI741756 |
| | subunit H” | |
| DKFZP566C243 | DKFZP566C243 protein | AL050274 |
| PPP1R3D | “protein phosphatase 1, regulatory subunit 3D” | Y18206 |
| SBA2 | CS box-containing WD protein | AF038187 |
| MEF2A | “MADS box transcription enhancer factor 2, polypeptide | U49020 |
| | A (myocyte enhancer factor 2A)” | |
| | | J05614 |
| UNC13 | unc-13-like (C. elegans) | AF020202 |
| HFL-EDDG1 | erythroid differentiation and denucleation factor 1 | AF048849 |
| LTA4H | leukotriene A4 hydrolase | J03459 |
| METTL1 | methyltransferase-like 1 | Y18643 |
| | | AD000092 |
| | “Homo sapiens cDNA FLJ40021 fis, clone | AL080094 |
| | STOMA2006904” | |
| IFIT1 | interferon-induced protein with tetratricopeptide repeats 1 | M24594 |
| TEF | thyrotrophic embryonic factor | U44059 |
| HMOX2 | heme oxygenase (decycling) 2 | AI086057 |
| DDB1 | “damage-specific DNA binding protein 1, 127 kDa” | U32986 |
| AKAP8 | A kinase (PRKA) anchor protein 8 | Y11997 |
| SLC9A1 | “solute carrier family 9 (sodium/hydrogen exchanger), | S68616 |
| | isoform 1 (antiporter, Na+/H+, amiloride sensitive)” | |
| ACADM | “acyl-Coenzyme A dehydrogenase, C-4 to C-12 straight | M91432 |
| | chain” | |
| NEURL | neuralized-like (Drosophila) | AF029729 |
| CDKN1B | “cyclin-dependent kinase inhibitor 1B (p27, Kip1)” | AI304854 |
| ASH2L | “ash2 (absent, small, or homeotic)-like (Drosophila)” | AB022785 |
| KHDRBS1 | “KH domain containing, RNA binding, signal transduction | M88108 |
| | associated 1” | |
| SNAP25 | “synaptosomal-associated protein, 25 kDa” | D21267 |
| RP2 | retinitis pigmentosa 2 (X-linked recessive) | AJ007590 |
| ACAT2 | acetyl-Coenzyme A acetyltransferase 2 (acetoacetyl | S70154 |
| | Coenzyme A thiolase) | |
| ATP6V1A1 | “ATPase, H+ transporting, lysosomal 70 kDa, V1 subunit | L09235 |
| | A, isoform 1” | |
| AQP1 | “aquaporin 1 (channel-forming integral protein, 28 kDa)” | U41518 |
| PPP1R8 | “protein phosphatase 1, regulatory (inhibitor) subunit 8” | U14575 |
| HLA-DOB | “major histocompatibility complex, class II, DO beta” | X03066 |
| ENSA | endosulfine alpha | X99906 |
| MXI1 | MAX interacting protein 1 | L07648 |
| PSMD4 | “proteasome (prosome, macropain) 26S subunit, non- | U51007 |
| | ATPase, 4” | |
| SLC6A2 | “solute carrier family 6 (neurotransmitter transporter, | X91117 |
| | noradrenalin), member 2” | |
| GTF2I | “general transcription factor II, i” | U77948 |
| | | M35093 |
| ZFP36L2 | “zinc finger protein 36, C3H type-like 2” | U07802 |
| NUP98 | nucleoporin 98 kDa | AF042357 |
| MYOZ3 | myozenin 3 | AF052497 |
| NF1 | “neurofibromin 1 (neurofibromatosis, von | D12625 |
| | Recklinghausen disease, Watson disease)” | |
| | Homo sapiens mRNA; cDNA DKFZp564O0122 (from | AL049951 |
| | clone DKFZp564O0122) | |
| PSMC2 | “proteasome (prosome, macropain) 26S subunit, | D11094 |
| | ATPase, 2” | |
| PPP3CB | “protein phosphatase 3 (formerly 2B), catalytic subunit, | M29551 |
| | beta isoform (calcineurin A beta)” | |
| ITGA2B | “integrin, alpha 2b (platelet glycoprotein IIb of IIb/IIIa | M34480 |
| | complex, antigen CD41B)” | |
| FGF18 | fibroblast growth factor 18 | AF075292 |
| PYCR1 | pyrroline-5-carboxylate reductase 1 | M77836 |
| EIF4B | eukaryotic translation initiation factor 4B | X55733 |
| KIAA0806 | KIAA0806 gene product | R93981 |
| | “Homo sapiens cDNA FLJ31348 fis, clone | AI970189 |
| | MESAN2000026” | |
| | | AC002073 |
| MGC5576 | hypothetical protein MGC5576 | W27939 |
| UBE2E1 | “ubiquitin-conjugating enzyme E2E 1 (UBC4/5 homolog, | AI039880 |
| | yeast)” | |
| JJAZ1 | joined to JAZF1 | D63881 |
| PMS1 | PMS1 postmeiotic segregation increased 1 (S. cerevisiae) | U13695 |
| KIAA0240 | KIAA0240 protein | D87077 |
| TBCD | tubulin-specific chaperone d | AJ006417 |
| NUP214 | nucleoporin 214 kDa | X64228 |
| FOSL2 | FOS-like antigen 2 | X16706 |
| PAFAH1B1 | “platelet-activating factor acetylhydrolase, isoform Ib, | L25107 |
| | alpha subunit 45 kDa” | |
| PSMA1 | “proteasome (prosome, macropain) subunit, alpha type, | M64992 |
| | 1” | |
| | ESTs | AI184710 |
| APOBEC3B | “apolipoprotein B mRNA editing enzyme, catalytic | AL022318 |
| | polypeptide-like 3B” | |
| | | U18671 |
| H41 | hypothetical protein H41 | H15872 |
| | | HG4582-HT4987 |
| ORC1L | “origin recognition complex, subunit 1-like (yeast)” | U40152 |
| XDH | xanthene dehydrogenase | U39487 |
| | Homo sapiens mRNA; cDNA DKFZp434M162 (from | W72239 |
| | clone DKFZp434M162) | |
| FUBP3 | far upstream element (FUSE) binding protein 3 | U69127 |
| ID1 | “inhibitor of DNA binding 1, dominant negative helix-loop- | S78825 |
| | helix protein” | |
| KIAA0637 | KIAA0637 gene product | AB014537 |
| CLTB | “clathrin, light polypeptide (Lcb)” | M20470 |
| KIAA1094 | KIAA1094 protein | AB029017 |
| RAB1A | “RAB1A, member RAS oncogene family” | M28209 |
| ERCC6 | “excision repair cross-complementing rodent repair | L04791 |
| | deficiency, complementation group 6” | |
| MYT1 | myelin transcription factor 1 | AB028973 |
| MGC10471 | hypothetical protein MGC10471 | X13956 |
| C12orf8 | chromosome 12 open reading frame 8 | X94910 |
| MSL3L1 | male-specific lethal 3-like 1 (Drosophila) | AL050178 |
| CSTF2T | likely ortholog of mouse variant polyadenylation protein | AB014589 |
| | CSTF-64 | |
| GS3955 | GS3955 protein | D87119 |
| | | U14573 |
| MTA1 | metastasis associated 1 | U35113 |
| FLJ20619 | hypothetical protein FLJ20619 | AL049431 |
| DNAJC7 | “DnaJ (Hsp40) homolog, subfamily C, member 7” | W28595 |
| TFRC | “transferrin receptor (p90, CD71)” | X01060 |
| KIAA0218 | KIAA0218 gene product | D86972 |
| KIAA1089 | KIAA1089 protein | AB029012 |
| FCGR2A | “Fc fragment of IgG, low affinity IIa, receptor for (CD32)” | M31932 |
| CSNK1A1 | “casein kinase 1, alpha 1” | L37042 |
| HPS1 | Hermansky-Pudlak syndrome 1 | U65676 |
| ACK1 | activated p21cdc42Hs kinase | L13738 |
| MAP-1 | modulator of apoptosis 1 | AI670788 |
| DDX9 | “DEAD/H (Asp-Glu-Ala-Asp/His) box polypeptide 9 (RNA | L13848 |
| | helicase A, nuclear DNA helicase II; leukophysin)” | |
| FAM8A1 | “family with sequence similarity 8, member A1” | AL050128 |
| PRO2730 | hypothetical protein PRO2730 | AL045811 |
| | Homo sapiens mRNA; cDNA DKFZp586H201 (from | AL049430 |
| | clone DKFZp586H201) | |
| KIAA0146 | KIAA0146 protein | D63480 |
| NUDEL | LIS1-interacting protein NUDEL; endooligopeptidase A | AF038203 |
| ARC | activity-regulated cytoskeleton-associated protein | D87468 |
| HMBS | hydroxymethylbilane synthase | M95623 |
| TRA1 | tumor rejection antigen (gp96) 1 | X15187 |
| | | U12471 |
| DAP | death-associated protein | X76105 |
| RYBP | RING1 and YY1 binding protein | AL049940 |
| RGS19 | regulator of G-protein signalling 19 | X91809 |
| BMP10 | bone morphogenetic protein 10 | AF101441 |
| KIAA0492 | KIAA0492 protein | AB007961 |
| URKL1 | uridine kinase-like 1 | AI249721 |
| SFRS2 | “splicing factor, arginine/serine-rich 2” | X75755 |
| CAPNS1 | “calpain, small subunit 1” | X04106 |
| C1orf8 | chromosome 1 open reading frame 8 | Z78368 |
| UBE3B | ubiquitin protein ligase | AI749193 |
| E2F3 | E2F transcription factor 3 | D38550 |
| | | J04423 |
| USP1 | ubiquitin specific protease 1 | AB014458 |
| TNRC15 | trinucleotide repeat containing 15 | AB014542 |
| IL5RA | “interleukin 5 receptor, alpha” | M75914 |
| | | X03453 |
| RHEB2 | Ras homolog enriched in brain 2 | D78132 |
| LSM6 | Sm protein F | AA917945 |
| TBX5 | T-box 5 | Y09445 |
| | Homo sapiens mRNA; cDNA DKFZp451N147 (from | AA534868 |
| | clone DKFZp451N147) | |
| ARSE | arylsulfatase E (chondrodysplasia punctata 1) | X83573 |
| LCP1 | lymphocyte cytosolic protein 1 (L-plastin) | J02923 |
| CSF1 | colony stimulating factor 1 (macrophage) | M37435 |
| DHCR7 | 7-dehydrocholesterol reductase | AF034544 |

Recent technical developments have now facilitated the analysis of large numbers of genes by means of the use of high density microarrays or “chips”. Each location on such a chip contains a sequence related to a specific sequence, such that when a signal (such as a visual color, produced by the use of suitable colored conjugate) is present, it can be readily related to the binding of sequences specific for a particular gene, the identity of which is determined by the position of the signal in the array. Suitable computer programs may then be used to analyze and present (in graphical and/or tabular form) the data extracted from the microarray signals. In addition to providing information relating to the expression of specific genes, high density microarrays may also be used to generate “fingerprints” which are characteristic of, for example, a particular disease, treatment response or (as in the case of the invention disclosed herein) prognostic group. The fingerprint thus obtained may be subjected to analysis by any of a number of statistical techniques (including cluster analysis, as described in the illustrative example, hereinbelow), in order to assign said fingerprint to a discrete results group. The results group may be one of a binary pair (such as the good prognosis/poor prognosis pair of the present invention), or it may be one of a more complex series of groups (such as in the case of the differential diagnosis of several pathological possibilities.)

Suitable high density microarrays may either be purchased “off-the-shelf”, pre-loaded with an array of oligonucleotide sequences (for example the Genechip Human Genome arrays produced by Affymetrix, Santa Clara, Calif., USA), or alternatively may be custom-produced such that they bear a subset of the total genome, wherein said subset is relevant for the desired diagnostic, prognostic or drug discovery application of the microarray. Many different materials and techniques may be used in the construction of high density microarrays, the details of which appear in many publications including U.S. Pat. No. 6,344,316, which is in its entirety incorporated herein by reference.

The techniques used to obtain, purify and hybridize RNA and other nucleic acids are varied and well known to all skilled artisans in the field. Details of many such suitable techniques are to be found in standard reference works such as the book “Molecular cloning: a laboratory manual” by Sambrook, J., Fritsch, E. F. & Maniatis, T., Cold Spring Harbor, N.Y., 2^{nd} ed., 1989 (and all later editions), which is incorporated herein by reference in its entirety.

In addition, Methods of isolating total mRNA are described in detail in Chapter 3 of Laboratory Techniques in Biochemistry and Molecular Biology: Hybridization with Nucleic Acid Probes, Part I. Theory and Nucleic Acid Preparation, P. Tijssen, ed. Elsevier, N.Y. (1993). More specific information related to the use of polymerase chain reaction (PCR) techniques may be gleaned from “Innis et al. eds., PCR Protocols: A guide to method and applications”, which is incorporated herein by reference.

Following isolation of the nucleic acids sequences and their purification and hybridization to a suitable high density chip, binding is determined by means of a suitable detection method. In a preferred embodiment, the hybridized nucleic acids are detected by detecting one or more labels attached to the sample nucleic acids. The labels may be incorporated by any of a number of means well known to those of skill in the art. Labels may be introduced either during the course of the synthesis of the nucleic acid sequences (e.g. during a PCR reaction) or as a discrete post-synthetic step. Detectable labels suitable for use in the present invention include any composition detectable by spectroscopic, photochemical, biochemical, immunochemical, electrical, optical or chemical means. Particularly preferred are labels such as biotin for staining with labeled streptavidin conjugate, magnetic beads (e.g., Dynabeads™), fluorescent dyes (e.g., fluorescein, texas red, rhodamine, green fluorescent protein, and the like (obtainable from Molecular Probes, Eugene, Oreg., USA). However, other label types, including radiolabels and enzymes may also be usefully employed.

Several different types of microarray may be used or produced in order to work the present invention. Thus, a variety of different substrate types, including (but not limited to) metal oxides, nylon, ceramic material and glasses may be used to construct the microarray. In a commonly-used configuration, the microarray is constructed such it has a surface area less than 6.25 cm², preferably in the range of about 1.6 cm² to 6.25 cm². Details of the construction of microarrays suitable for use in the present invention are now well known in the art, and may be obtained from a variety of publications including the aforementioned U.S. Pat. No. 6,344,316, U.S. Pat. No. 6,232,068 and U.S. Pat. No. 5,510,270, all of which are incorporated herein in their entirety.

The following example is provided for illustrative purposes and in order to more particularly explain and describe the present invention. The present invention, however, is not limited to the particular embodiments disclosed in the example.

### Example 1

### Prognosis Determination by Means of Genetic Profiling of Tumor Material Obtained from ES Patients

### Methods

### Patient Samples

Fourteen primary tumor specimens and six metastases were obtained from 18 ES patients with non-metastatic disease. In the case of one patient, both primary and recurrent tumors were analyzed (SA37 and SA43), and two metastases were taken from another patient, six years apart (SA45 and SA46). All patients were admitted to the Pediatric Hematology Oncology Department at Schneider Children's Medical Center, Petach Tikva, Israel. Informed consent was obtained from the patients or their guardians, and the local and National Ethics Committees approved the research project. All patients were treated with a combination of aggressive chemotherapy, radiotherapy and surgery. Median age at diagnosis was 15 years (range 7-27). Five patients were females and 13 were males. Response to therapy was defined by histopathological response and assessed by percentage of tumor necrosis at the time of surgery (limb salvage procedure) following neoadjuvant chemotherapy and radiotherapy. Median follow up was 72.5 months (range 7-171). Tumors were snap-frozen in liquid nitrogen immediately after surgery and stored at −80° C. until use.

### Microarray Hybridization

Ten μg of total RNA was extracted from each tumor using Tri Reagent (Molecular Research Center, Inc. Cincinnati, Ohio). Double stranded cDNA was generated from 10 ug of total RNA using the SuperScript Choice System from Gibco Brl (Rockville, Md., USA), using an oligo(dT)₂₄ primer containing a T7 promoter site at the 3′ end (Genset, La Jolla, Calif.). cDNAs were purified via a phenol-chloroform extraction followed by an ethanol precipitation. Purified cDNA was used as template for In vitro transcription (IVT), which was performed with T7 RNA polymerase and biotin-labeled ribonucleotides, using the ENZO BioArray High Yield RNA Transcript Labeling Kit (Enzo Diagnostics, New York, N.Y.). Labeled in vitro transcripts were purified over RNeasy mini columns (Qiagen, Valencia, Calif.) according to manufacturer's instructions. The labeled cRNA was fragmented at 94° C. for 35 min in fragmentation buffer (40 mM Tris-acetate, pH 8.1/100 mM potassium acetate, 30 mM magnesium acetate), and a hybridization mix was generated by addition of herring sperm DNA (0.1 mg/ml), acetylated BSA (0.5 mg/ml, Invitrogen), sodium chloride (1 M), Tris-acetate (10 mM), and Tween-20 (0.0001%). A mixture of four control bacterial and phage cRNA (1.5 pM BioB, 5 pM BioC, 25 pM BioD, and 100 pM Cre) was included to serve as an internal control for hybridization efficiency.

Aliquots of each sample (12 μg cRNA in 200 μl hybridization mix) were hybridized to a Genechip Human Genome U95Av2 array (Affymetrix, Santa Clara, Calif., USA). After hybridization, each array was washed according to procedures developed by the manufacturer (Affymetrix), and stained with streptavidin-phycoerythrin conjugate (Molecular Probes, Eugene, Oreg.). The hybridization signal was amplified by using biotinylated anti-streptavidin antibodies (Vector Laboratories, Burlingame, Calif.), followed by restaining with streptavidin phycoerythrin. Arrays were scanned by the GeneArray scanner G2500A (Hewlett Packard, Palo Alto, Calif.), and scanned images were visually inspected for hybridization imperfections. Arrays were analyzed using Genechip 4.1 software (Affymetrix). The expression value for each gene was determined by calculating the average differences of the probe pairs in use for that gene.

Two samples were analyzed in duplicate and results were reproducible.

### Data Analysis:

### Normalization and Filtering

The microarray results were analyzed using the GeneSpring Software®. Normalization was performed by setting expression values lower than zero to zero and than each measurement was divided by the median of all measurements in that sample.

In order to filter out genes that are not expressed in any of the groups, Affymetrix absolute call (MAS 4.0: P, M—expressed genes, A—not expressed) was used. Genes that were expressed in one group were defined as genes expressed in at least 3 samples.

### Selecting for Differentially Expressed Genes

A Student's t-test was applied for each gene, and genes with an adjusted P-value less then 0.01 were selected as differentially expressed genes. P-values were corrected to reduce false positive using Benjamini and Hochberg False Discovery Rate (Benjamini, Y. et al. J. Roy. Stat. Soc. B., 57, 289-300 (1995)].

### Hierarchical Clustering

Divisive hierarchical clustering [Everitt, B. S. Cluster analysis. 3^{rd} edition, 62-65 (Arnold, London, 1993)) was performed as described by Eiesen et al. [Eisen, M. B. et al. Proc. Natl. Acad. Sci. USA 95, 14863-14868 (1998], using centered correlation as the measurement distance.

### Progression Free Survival Analysis

Kaplan-Meier progression free survival analysis, using the log rank test, was performed in order to correlate the microarray classification results with patients' clinical outcome.

### Quantitative Real Time PCR (RQ-PCR)

The microarray derived expression data was evaluated for the cadherin-11 and MTA1 genes using quantitative PCR by the LightCycler system (Roche Diagnostics, Manheim, Germany). cDNA was prepared using the Reverse Transcription System (Promega Corporation, Madison, Wis.) and purified with GFX PCR DNA and Gel. Band Purification kit (Amersham Biosciences, Piscataway, N.J.). 5 μl was amplified in a 20 μl reaction containing 4 mM MgCL₂, 5 μM of each primer and LightCycler—FastStart DNA Master SYBR Green I mix (Roche Diagnostics).

Cadherin-11 primers: sense 5′-AGAGGCCTACATTCTGAACG-3′ and
antisense 5′-TTCTTTCTTTTGCCTTCTCAGG-3′. MTA1 primers:
sense 5′-AGCTACGAGCAGCACAACGGGGT-3′ and
antisense 5′-CACGCTTGGTTTCCGAGGAT-3′.

All examinations were performed in duplicate and data analysis was done using the LightCycler Software.

### Results

The study included 14 tumor samples from localized ES patients. The gene expression profile of 7 tumors from patients who had progressed between 5 months up to 5 years from diagnosis (defined as High Risk—HR) was compared with 7 tumors from patients who were disease free for a long period of follow up (median 92 months; range 66-171) (defined as Low Risk—LR).

In brief, RNA was isolated from each tumor and hybridized to Affymetrix oligonucleotide high-density arrays U95Av2. A subset of genes that distinguish between the two groups (HR and LR) by two steps was identified. Firstly, 8098 genes that were expressed in one of the groups, in at least 3 samples, were selected. Subsequently, 818 genes differentially expressed in either the HR or the LR groups (t-test; P<0.01) were studied. These 818 most significant genes are listed in Table 1, hereinabove.

In order to control false positive results as a consequence of multiple comparisons, the P values were adjusted using the False Discovery Rate (FDR) method [Everitt, B. S. Cluster analysis. 3^{rd} edition, 62-65 (Arnold, London, Benjamin, Y. et al., J. Roy. Stat. Soc. B, 57, 289-300 (1995)].

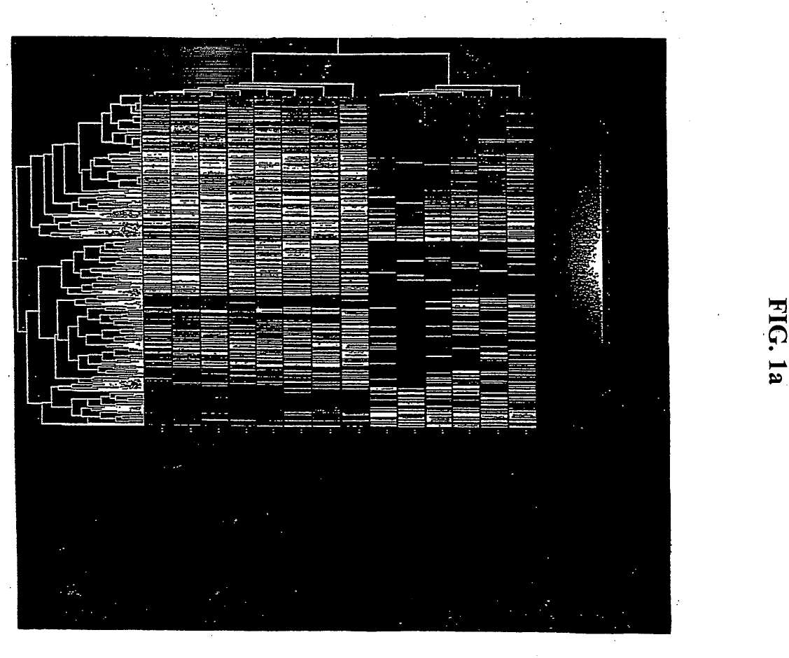

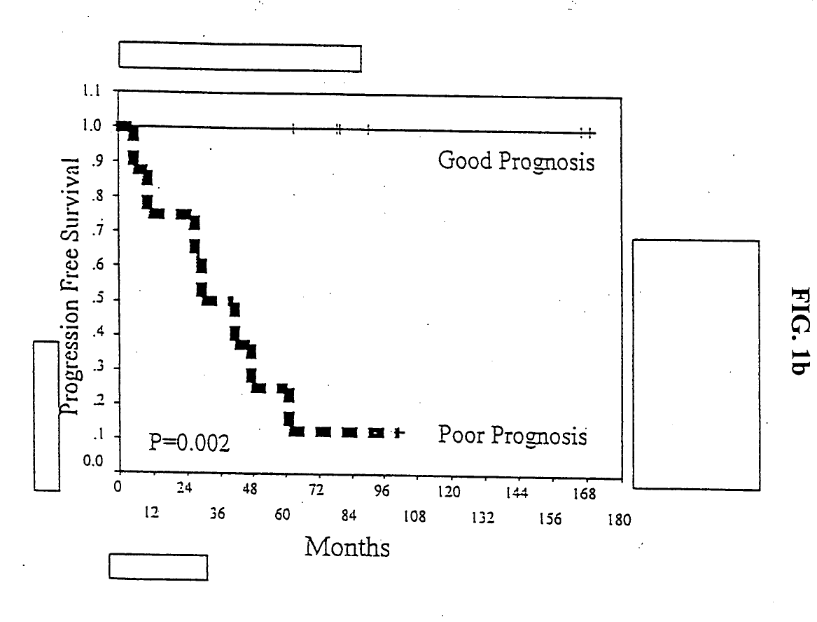

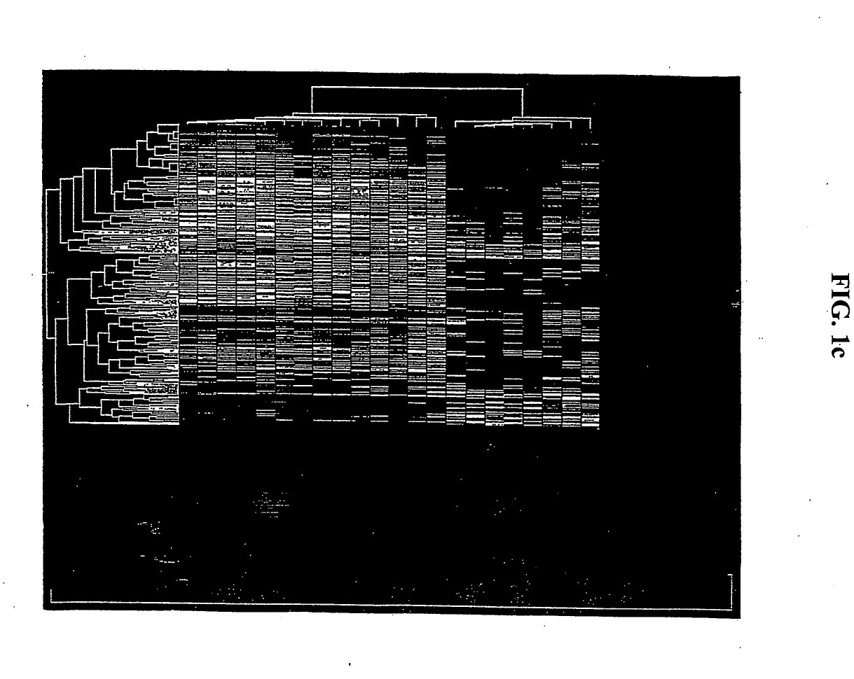

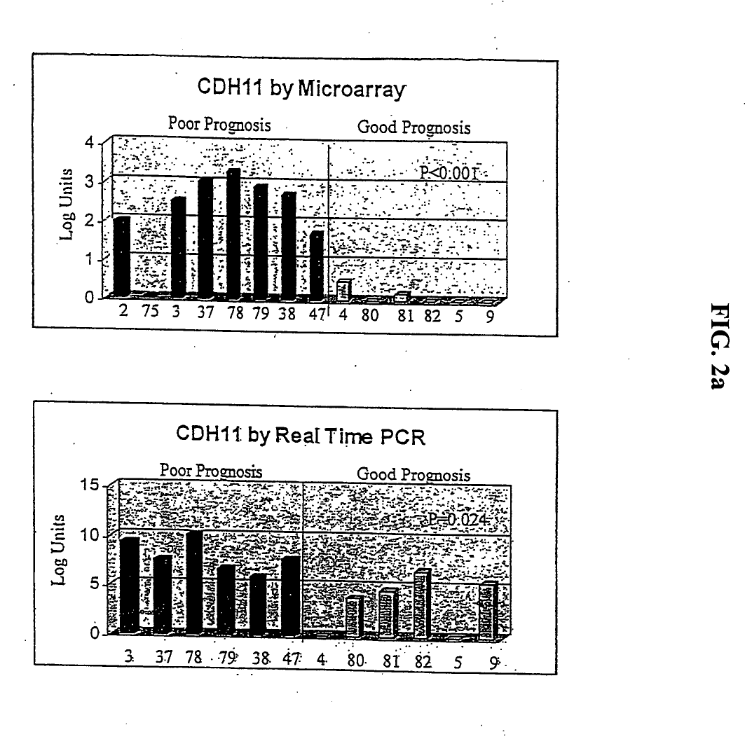

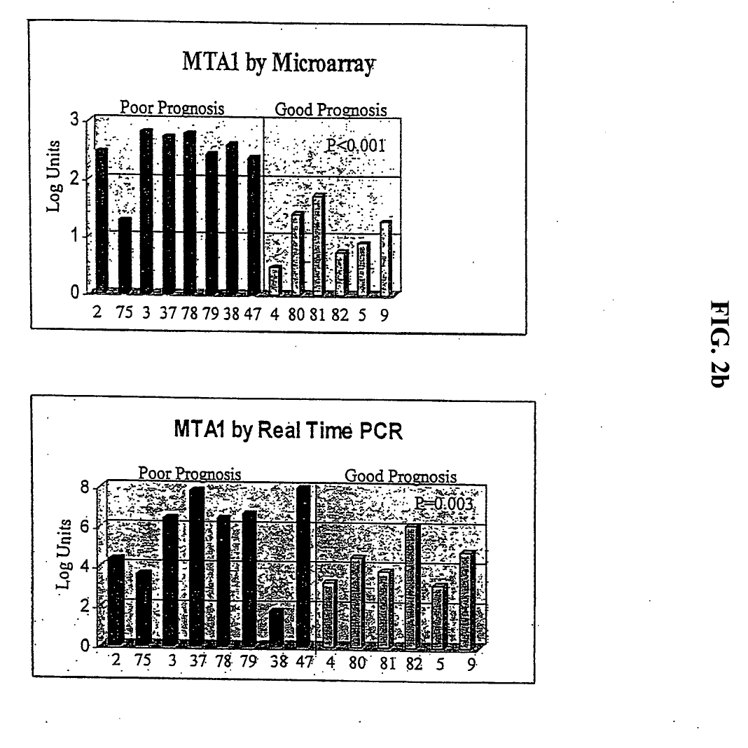

Using hierarchical clustering, based on the 818 genes, for prognosis profile, two distinct clusters could be determined: poor and good prognosis signatures (FIG. 1a). All of the seven HR and six out of the seven LR patients (86%) were classified as poor and good prognosis signatures, respectively (Table 2). One clinically LR patient who was disease free for a long period of follow up (97 months), was classified in the poor prognosis signature group. Each one of the 818 genes is sufficient for the prediction of prognosis.

TABLE 2
Clinical data, disease course and results of
molecular classification

| | | | | | | Microarray |
|---|---|---|---|---|---|---|
| | | | Response | | | classification |
| | Age | Primary | to therapy | Relapse | Outcome | prognosis |
| Sample | (years) | Site | % necrosis | (months) | (months) | group |
| High Risk | | | | | | |
| SA3 | 21 | Pelvis | <90% | Local (5) | EX (7) | Poor |
| SA37 | 7 | Cranium | N.D | Local (29) | EX (44) | Poor |
| SA38 | 17 | Pelvis | <90% | Local (10) | EX (18) | Poor |
| SA47 | 20 | Pelvis | >90% | Cranium (61) | AWD (76) | Poor |
| SA75 | 18 | Pelvis | <90% | Local (27) | EX (49) | Poor |
| SA78 | 24 | Femur | <90% | Lung (47) | EX (65) | Poor |
| SA79 | 12 | Pelvis | >90% | Bone (41) | EX (60) | Poor |
| Low Risk | | | | | | |
| SA2 | 15 | Pelvis | >90% | — | NED (103) | Poor |
| SA4 | 14 | Chest | N.D | — | NED (92) | Good |
| SA5 | 13 | Radius | <90% | — | NED (66) | Good |
| SA9 | 13 | Tibia | >90% | | NED (168) | Good |
| SA80 | 15 | Pelvis | >90% | — | NED (81) | Good |
| SA81 | 14 | Pelvis | >90% | — | NED (82) | Good |
| SA82 | 11 | Tibia | >90% | | NED (173) | Good |
| Metastases | | | | | | |
| SA43 | 7 | Cranium | N.D | Local (29) | EX (44) | Poor |
| SA44 | 27 | Femur | >90% | Lung (61) | NED (91) | Good |
| SA45 | 16 | Femur | <90% | Brain (128) | AWD (151) | Poor |
| SA46 | 16 | Femur | <90% | Lung (67) | AWD (151) | Poor |
| SA76 | 20 | Pelvis | <90% | Lung (24) | EX (44) | Poor |
| SA77 | 8 | Pelvis | <90% | Local (37) | EX (104) | Good |

EX = Expired;
NED = No Evidence of Disease;
AWD = Alive With Disease
Numbers in brackets = time from diagnosis;
N.D = not done

Kaplan-Meier life table analysis indicated that the patients predicted to have a good prognosis signature had a significantly improved progression free survival (PFS) compared with those predicted to have a poor prognosis signature (FIG. 1b, P=0.002).

Additionally, the genes were reordered into 2 major clusters that were divided into 6 sub-clusters, by performing hierarchical clustering of all signature genes (FIG. 1c). The two major groups correspond to (i) over-expressed in the poor prognosis group and down-regulated in the good prognosis group, and (ii) vice versa. The six sub-clusters correspond to the variability of genes among the patients with poor or good prognosis signatures, which was more considerable in the poor prognosis group. Genes that were over-expressed in the poor prognosis patients include known markers of ES like EWS breakpoint region 1 and beta 2 microglobulin, genes regulating the cell cycle like CDK2, E2F, RAF and MAPKs, and genes associated with invasion and metastasis like cadherin-11 and MTA1. The last two belong to subclusters 5 and 6, genes which were homogeneously expressed in all patients. Down-regulated genes in the poor prognosis patients, included tumor suppressor genes like FHIT and LLGL1, genes inducing apoptosis like TNFRSF12, TGFB1, CASP10 and TP63 and inhibitors of angiogenesis like IFIT1 and IRF2.

Two genes that were significantly over expressed in the poor prognosis signature group (p<0.01) are of particular interest; both are associated with invasion and metastasis. The first one is cadherin11 (OB-cadherin), a homophilic calcium-dependent cell adhesion molecule, and the second is MTA1, tumor metastasis-associated gene. Cadherins modulate calcium ion-dependent cell-cell adhesion and are important in cell aggregation, migration and sorting. Defective cell-cell and cell-matrix adhesion are among the hallmarks of cancer. Disruption of the cadherin-catenin complex has been demonstrated in carcinomas arising in several tissues including prostate, gastric and breast carcinomas, and has been correlated with various pathologic and clinical features, such as tumor differentiation, proliferation and a poor patient prognosis.

The MTA1 gene is a novel, highly conserved gene that encodes a nuclear protein product. Examination of the MTA1 protein suggests that it is a histone deacetylase and may serve multiple functions in cellular signaling, chromosome remodeling and transcription processes that are important in the progression, invasion and growth of metastatic cells. The gene has been found to be over-expressed in a variety of human cell lines (breast, ovarian, lung, gastric and colorectal) and cancerous tissues (breast, esophageal, colorectal, gastric and pancreatic cancer).

To validate the microarray data, these two over-expressed genes were analyzed in further detail using reverse transcriptase—quantitative Real Time PCR (RQ-PCR). Microarray-based expression and RQ-PCR based expression data correlated significantly (FIGS. 2a and b). The mean log expression value of the poor prognosis signature group is significantly higher than that of the good prognosis signature group for both genes, cadherin-11 and MTA1, P=0.024 and P=0.003, respectively.

Six metastases from localized patients who progressed were further tested, using the unsupervised learning methodology, whether the poor and good prognosis signature set of genes can classify metastatic tissues to one of the prognostic groups, or as a distinct group.

While specific embodiments of the invention have been described for the purpose of illustration, it will be understood that the invention may be carried out in practice by skilled persons with many modifications, variations and adaptations, without departing from its spirit or exceeding the scope of the claims.

1. A method for assessing the prognosis of Ewing's Sarcoma (ES) patients comprising determining the expression pattern of a defined set of genes in tumor material obtained from said patients, and assigning said expression pattern to either a good prognosis or poor prognosis group.

2. The method according to claim 1, wherein the expression pattern of the aforementioned defined set of genes is determined by means of a technique selected from the group consisting of nucleic acid hybridization, semi-quantitative RT-PCR, quantitative real time RT-PCR, immunohistochemistry and ELISA.

3. The method according to claim 2, wherein the expression pattern of the aforementioned defined set of genes is determined by means of a nucleic acid hybridization technique.

4. The method according to claim 3, wherein the nucleic acid hybridization technique comprises the steps of extracting total RNA from the ES-patient tumor material, generating double-stranded cDNA from said total RNA, performing in vitro transcription of said cDNA, labeling the RNA transcript obtained thereby, hybridization of said RNA transcript to a solid-state human genome microarray.

5. The method according to claim 1, wherein the assignment of the gene expression pattern to one of the good or poor prognosis groups is performed by means of a hierarchical clustering technique.

6. The method according to claim 1, wherein the defined set of genes comprises genes selected from a group of 818 genes listed in Table 1, hereinabove.

7. The method according to claim 6, wherein the defined set of genes consists of between 1 and 100 genes selected from the group of 818 genes.

8. The method according to claim 6, wherein the defined set of genes consists of between 101 and 200 genes selected from the group of 818 genes.

9. The method according to claim 6, wherein the defined set of genes consists of between 201 and 300 genes selected from the group of 818 genes.

10. The method according to claim 6, wherein the defined set of genes consists of between 301 and 400 genes selected from the group of 818 genes.

11. The method according to claim 6, wherein the defined set of genes consists of between 401 and 500 genes selected from the group of 818 genes.

12. The method according to claim 6, wherein the defined set of genes consists of between 501 and 600 genes selected from the group of 818 genes.

13. The method according to claim 6, wherein the defined set of genes consists of between 601 and 700 genes selected from the group of 818 genes.

14. The method according to claim 6, wherein the defined set of genes consists of between 701 and 818 genes selected from the group of 818 genes.

15. A solid-state nucleic acid microarray comprising at least two nucleic acids affixed to a substrate, wherein each of said at least two nucleic acids consists of a partial sequence of one of the genes present in the group of 818 genes listed in Table 1, hereinabove.

16. The solid-state nucleic acid microarray according to claim 15 comprising 818 nucleic acid sequences, wherein each of said sequences consists of a partial sequence of one of the 818 genes listed in Table 1, hereinabove.

17. The solid-state nucleic acid microarray according to claim 15 further comprising one or more control nucleic acid sequences.

18. A kit comprising a solid-state nucleic acid microarray according to claim 15, together with an instruction sheet.
